(19) United States
(12) Patent Application Publication
(10) Pub. No.: US 2022/0397572 A1
(43) Pub. Date: Dec. 15, 2022

(54) BEAD SYSTEMS, METHODS, AND APPARATUS FOR MAGNETIC BEAD-BASED ANALYTE DETECTION

(71) Applicant: Quantum Diamond Technologies Inc., Somerville, MA (US)

(72) Inventors: David R. Glenn, Cambridge, MA (US); Colin B. Connolly, Cambridge, MA (US); Jeffrey D. Randall, Westford, MA (US)

(21) Appl. No.: 17/776,068

(22) PCT Filed: Nov. 3, 2020
(86) PCT No.: PCT/US20/58664

§ 371 Date: May 11, 2022

Related U.S. Application Data
(60) Provisional application No. 62/934,197, filed on Nov. 12, 2019.

(51) Int. Cl. G01N 33/543

(52) U.S. Cl. CPC G01N 33/54333

(57) ABSTRACT

The present application discloses methods and apparatus for detecting a complex including an analyte that include contacting a sample solution containing an analyte with a population of functionalized superparamagnetic beads, which are functionalized to include a first moiety that associates with the analyte under suitable conditions, and contacting the sample solution with a population of functionalized ferromagnetic beads, which are functionalized to include a second moiety. Contact results in formation of complexes detectable by co-localization of the functionalized superparamagnetic bead and the functionalized ferromagnetic bead. Contact between a sample not containing the analyte in a sample solution, results in a magnetic interaction energy Dint between the functionalized superparamagnetic beads and the functionalized ferromagnetic beads, the magnetic interaction energy Dᵢₙₜ being less than or equal to 5k_{B}T, where k_{B} is the Boltzmann constant and T is the temperature of the sample solution.

### CROSS REFERENCE TO RELATED APPLICATION

This application claims priority from U.S. Provisional Patent Application No. 62/934,197 filed on 12 Nov. 2019 entitled BEAD SYSTEMS, METHODS, AND APPARATUS FOR MAGNETIC BEAD-BASED ANALYTE DETECTION, which is hereby incorporated by reference.

### BACKGROUND

Enzyme linked immunosorbent assay (ELISA) has been an industry-wide standard research technique used for measuring protein analytes from biological matrices since its introduction in the 1960s. In its basic conception, two antibodies (immunoglobulins) are used to capture a single protein analyte. The resulting immunocomplex is identified and measured using an enzyme and reporter buffer. The enzyme is typically bound to one of the antibodies through covalent bonding. The enzyme, when incubated in the presence of the reporter buffer, converts the substrate to a functional reporter which can be measured analytically by spectrophotometric means.

Current state of the art ELISA-related technologies are replacing the plate-based format with a single-bead-based format. Single-bead-based ELISAs have one antibody that is bound to a solid surface, typically a bead, and a second antibody that is labeled with biotin. The capture bead contains material that allows it to be easily manipulated by an applied magnetic field, including separating the bead and any analytes bound to the bead from a sample suspension. This process, called magnetic separation, is well known in the art and can be used to concentrate the target analyte and to remove unbound material such as unwanted proteins that can contribute to signal background. Compared to traditional ELISAs, single-bead-based ELISAs can provide improved sensitivity to target analytes with a lower background in a shorter amount of time.

Magnetic multi-bead assays make use of distinct bead types to determine analyte concentration in a sample by detecting the formation of bead complexes that are bound by the target analyte. Some magnetic bead assays, such as assays described in PCT Patent Application No. PCT/US2017/068126 filed on Dec. 22, 2017 and entitled METHODS AND APPARATUS FOR MAGNETIC MULTI-BEAD ASSAYS that is incorporated by reference herein, rely on detecting target-specific binding between two distinguishable magnetic bead types, a superparamagnetic bead A and a ferromagnetic bead B. Bead A is coated with antibodies of type X specific to the target analyte (e.g., an antigen), while bead B is coated with antibodies of type Y specific to a different portion of the same target analyte. In the presence of the target analyte, bead A and bead B become bound together by each binding to the same target analyte, forming a complex, such as an immunocomplex. In some magnetic bead-based assays, a wide-field diamond magnetic imaging apparatus, described in PCT Patent Application No. PCT/US2017/057628 filed on Oct. 20, 2017 and entitled METHODS AND APPARATUS FOR MAGNETIC PARTICLE ANALYSIS USING DIAMOND MAGNETIC IMAGING that is incorporated by reference herein, identifies complexes by co-localization of bead A and bead B, differentiating them from unbound bead A or bead B, as well as from aggregates containing only bead A or bead B (“A-like” or “B-like” aggregates).

In the absence of the target analyte, the assay should detect zero complexes. The sensitivity of the assay depends on this null measurement, so that detecting a very small number (e.g., as low as one) of complexes may be conclusively associated with a nonzero target analyte concentration in the sample. To that end, bead A and bead B must not form bound complexes in the absence of the target analyte. The magnetic imaging assay only detects co-localization of bead A and bead B—not the target analyte directly—and therefore cannot distinguish analyte-containing bound complexes from false-positive bound complexes not containing the target analyte.

One mechanism for forming bound complexes in the absence of the target analyte is attractive magnetic interactions between bead A and bead B. Since the beads A and B are superparamagnetic and ferromagnetic, respectively, and since they must be sufficiently magnetic to be detected, attractive magnetic interactions between the beads will generally always be present to some extent. However, to form a stably bound complex, the magnetic attraction has to be strong enough to resist forces that drive the beads apart, such as the ever-present random thermal fluctuations of Brownian motion in the sample solution.

Therefore, there is a need for continuing improvement in sensitivity and specificity to target analytes in magnetic bead-based assays.

### BRIEF SUMMARY

Various embodiments disclosed herein relate to methods and apparatus for detecting a complex including an analyte in a sample by observing complexes containing two or more distinguishable beads (i.e., beads having distinguishable magnetic properties) that are bound by the analyte. In accordance with one or more embodiments, a bead system for bead-based analyte detection includes a plurality of functionalized superparamagnetic beads having a diameter d_{A} and a volume magnetic susceptibility X_{A}. The functionalized superparamagnetic beads are functionalized to include a first moiety that associates with an analyte under suitable conditions in a sample solution. The system further includes a plurality of functionalized ferromagnetic beads having a diameter d_{B} and a magnetic dipole moment p_{B}. The functionalized ferromagnetic beads are functionalized to include a second moiety that associates with the analyte under suitable conditions in the sample solution. Contact between a sample containing the analyte in the sample solution, the functionalized superparamagnetic beads, and the functionalized ferromagnetic beads results in formation of complexes, each including one of the functionalized superparamagnetic beads, the analyte, and one of the functionalized ferromagnetic beads, the analyte detectable by co-localization of the functionalized superparamagnetic bead and the functionalized ferromagnetic bead. Contact between a sample not containing the analyte in a sample solution, the functionalized superparamagnetic beads, and the functionalized ferromagnetic beads results in a magnetic interaction energy Uᵢₙₜ between the functionalized superparamagnetic beads and the functionalized ferromagnetic beads, the magnetic interaction energy Uᵢₙₜ being less than or equal to 5k_{B}T, where k_{B} is the Boltzmann constant and T is the temperature of the sample solution. In some embodiments, the diameter d_{A} can be in a range of between about 0.1 μm and about 10 μm, such as in a range of between about 0.3 μm and about 3 μm, or preferably in a range of between about 0.5 μm and about 2 μm, or more preferably about 1 μm. In certain embodiments, the volume magnetic susceptibility X_{A} can be in a range of between about 0.01 and about 10, such as in a range of between about 0.1 and about 5, or preferably in a range of between about 0.5 and about 3, or more preferably about 1.37. In some embodiments, the diameter d_{B} can be in a range of between about 0.1 μm and about 10 μm, such as in a range of between about 0.3 μm and about 3 μm, or preferably in a range of between about 0.5 μm and about 2 μm, or more preferably about 1.8 μm. In certain embodiments, the magnetic dipole moment p_{B} can be in a range of between 0.02·(d_{B}/[μm])³ mA·μm² and

$${( \frac{20 {π}^{2} ( {k}_{B} T / [ J ] ) {( {d}_{A} / [ μ m ] )}^{3}}{( {μ}_{0} / [ \frac{J}{m {A}^{2} μ m} ] ) {X}_{A} Q} )}^{\frac{1}{2}} m A \cdot μ {m}^{2} ,$$

preferably between 0.2·(d_{B}/[μm])³ mA·μm² and

$${( \frac{20 {π}^{2} ( {k}_{B} T / [ J ] ) {( {d}_{A} / [ μ m ] )}^{3}}{( {μ}_{0} / [ \frac{J}{m {A}^{2} μ m} ] ) {X}_{A} Q} )}^{\frac{1}{2}} m A \cdot μ {m}^{2} ,$$

where μ₀ is the vacuum permeability, k_{B} is the Boltzmann constant, T is the temperature of the sample solution, and numerical values of Q(d_{A},d_{B}) are tabulated in Table 1, or more preferably about 1.0 mA·μm². In a specific embodiment, the diameter d_{A} can be in a range of between about 0.1 μm and about 10 μm, the volume magnetic susceptibility X_{A} can be in a range of between about 0.01 and about 10, the diameter d_{B} can be in a range of between about 0.1 μm and about 10 μm, and the magnetic dipole moment p_{B} can be in a range of between 0.02·(d_{B}/[μm])³ mA·μm² and

$${( \frac{20 {π}^{2} ( {k}_{B} T / [ J ] ) {( {d}_{A} / [ μ m ] )}^{3}}{( {μ}_{0} / [ \frac{J}{m {A}^{2} μ m} ] ) {X}_{A} Q} )}^{\frac{1}{2}} m A \cdot μ {m}^{2} ,$$

where μ₀ is the vacuum permeability, k_{B} is the Boltzmann constant, T is the temperature of the sample solution, and numerical values of Q(d_{A},d_{B}) are tabulated in Table 1. In some embodiments, each of the functionalized superparamagnetic beads can include a nonmagnetic core and superparamagnetic material distributed substantially uniformly around the nonmagnetic core. In other embodiments, each of the functionalized superparamagnetic beads can include superparamagnetic material distributed substantially uniformly throughout a volume of the functionalized superparamagnetic bead. In some embodiments, each of the functionalized ferromagnetic beads can include ferromagnetic material concentrated at a core of the functionalized ferromagnetic bead. In other embodiments, each of the functionalized ferromagnetic beads can include ferromagnetic material distributed substantially uniformly throughout a volume of the functionalized ferromagnetic bead. In still other embodiments, each of the functionalized ferromagnetic beads can include ferromagnetic material distributed over a surface of the functionalized ferromagnetic bead. In some embodiments, each of the functionalized superparamagnetic beads and/or the functionalized ferromagnetic beads can further include a nonmagnetic buffer layer around a surface of the bead. In certain embodiments, each of the first and the second moiety can be a receptor, protein, antibody, cell, virus, or nucleic acid sequence.

In accordance with one or more embodiments, a system for detecting a complex including an analyte includes a bead system including a plurality of functionalized superparamagnetic beads having a diameter d_{A} and a volume magnetic susceptibility X_{A}. The functionalized superparamagnetic beads are functionalized to include a first moiety that associates with an analyte under suitable conditions in a sample solution. The system further includes a plurality of functionalized ferromagnetic beads having a diameter d_{B} and a magnetic dipole moment p_{B}. The functionalized ferromagnetic beads are functionalized to include a second moiety that associates with the analyte under suitable conditions in the sample solution. Contact between a sample containing the analyte in the sample solution, the functionalized superparamagnetic beads, and the functionalized ferromagnetic beads results in formation of complexes, each including one of the functionalized superparamagnetic beads, the analyte, and one of the functionalized ferromagnetic beads, the analyte detectable by co-localization of the functionalized superparamagnetic bead and the functionalized ferromagnetic bead. Contact between a sample not containing the analyte in a sample solution, the functionalized superparamagnetic beads, and the functionalized ferromagnetic beads results in a magnetic interaction energy Uᵢₙₜ between the functionalized superparamagnetic beads and the functionalized ferromagnetic beads, the magnetic interaction energy Uᵢₙₜ being less than or equal to 5k_{B}T, where k_{B} is the Boltzmann constant and T is the temperature of the sample solution. The system further includes a detection apparatus for detecting complexes including the analyte by detecting co-localization of the functionalized superparamagnetic beads and the functionalized ferromagnetic beads. In some embodiments, the detection apparatus can include a substrate, on which the sample can be placed, the substrate including at least one optically detected magnetic resonance (ODMR) center, a light source configured to generate incident light that excites electrons within the at least one ODMR center from a ground state to an excited state, and a magnet for applying a bias magnetic field on the complex disposed over the at least one ODMR center. In these specific embodiments, the system further includes a microwave source configured to generate a microwave field incident on the at least one ODMR center, the microwave source being further configured to generate the microwave field with frequencies that correspond to ground state transitions in the at least one ODMR center, in which the at least one ODMR center produces emitted light when illuminated by the incident light, characteristics of the emitted light being influenced by the microwave field and by the magnetic functionalized beads associated with the analyte in the complex, and an optical photodetector that detects light emitted by the at least one ODMR center. In some embodiments, the at least one ODMR center can be a silicon vacancy center in a silicon carbide lattice in the substrate. In other embodiments, the at least one ODMR center can be a silicon vacancy center in a diamond lattice in the substrate. In still other embodiments, the at least one ODMR center can be a nitrogen-vacancy center in a diamond lattice in the substrate. In certain embodiments, the at least one ODMR center can be formed in an upper surface of the substrate. In some of these embodiments, the at least one ODMR center can be a plurality of ODMR centers formed in the upper surface of the substrate. In these embodiments, the optical photodetector can be an optical imaging system having an imaging sensor that images the emitted light from the plurality of ODMR centers.

In accordance with one or more embodiments, a method of detecting a complex including an analyte includes contacting a sample solution potentially containing an analyte with a population of functionalized superparamagnetic beads, which are functionalized to include a first moiety that associates with the analyte under suitable conditions, and contacting the sample solution with a population of functionalized ferromagnetic beads, which are functionalized to include a second moiety that associates with the analyte under suitable conditions.

Contact of the sample solution with the population of functionalized superparamagnetic beads and the population of functionalized ferromagnetic beads results in formation of complexes, when the analyte is present in the sample solution, each complex including one of the functionalized superparamagnetic beads, the analyte, and one of the functionalized ferromagnetic beads, or a magnetic interaction energy Uᵢₙₜ between the functionalized superparamagnetic beads and the functionalized ferromagnetic beads in the absence of the analyte in the sample solution, the magnetic interaction energy Uᵢₙₜ being less than or equal to 5k_{B}T, where k_{B} is the Boltzmann constant and T is the temperature of the sample solution. The method further includes detecting the complex including the analyte by detecting co-localization of the functionalized superparamagnetic bead and the functionalized ferromagnetic bead. In some embodiments, the method can further include applying a magnetic field gradient to the sample solution after contacting the sample with the population of functionalized superparamagnetic beads. In other embodiments, applying the magnetic field gradient to the sample solution can be performed after contacting the sample solution with the population of functionalized ferromagnetic beads. In some embodiments, the population of functionalized superparamagnetic beads and the population of functionalized ferromagnetic beads can be added to the sample solution sequentially. In other embodiments, the population of functionalized superparamagnetic beads and the population of functionalized ferromagnetic beads can be added to the sample solution simultaneously.

In certain embodiments, the method can further include applying a magnetic field gradient to the sample solution after contacting the sample solution with the functionalized superparamagnetic and ferromagnetic beads. In some embodiments, the method can further include varying the magnetic field gradient applied to the sample solution. In certain embodiments, the method can further include concentrating the sample solution after contacting the sample solution with the population of functionalized ferromagnetic beads. In some embodiments, the method can further include agglomerating a plurality of functionalized superparamagnetic and ferromagnetic beads, after contacting the sample solution with the population of functionalized ferromagnetic beads, before detecting the complex. In certain embodiments, detecting the complex can further include disposing the sample solution potentially including the complex over a substrate that includes at least one optically detected magnetic resonance (ODMR) center formed in the substrate. In these embodiments, detecting the complex further includes exciting electrons within the at least one ODMR center from a ground state to an excited state with incident light, applying a bias magnetic field on the complex, generating a microwave field incident on the at least one ODMR center, the microwave field including frequencies that correspond to ground state transitions in the at least one ODMR center, and analyzing light emitted by the at least one ODMR center, characteristics of the emitted light being influenced by the microwave field and by the functionalized superparamagnetic and ferromagnetic beads associated with the analyte in the complex. In some embodiments, the at least one ODMR center can be a nitrogen-vacancy center in a diamond lattice in the substrate. In certain embodiments, the at least one ODMR center can be formed in an upper surface of the substrate. In some embodiments, the at least one ODMR center can be a plurality of ODMR centers formed in the upper surface of the substrate. In certain embodiments, analyzing light emitted by the plurality of ODMR centers can include imaging the emitted light. In some embodiments, the method can further include dehydrating the sample solution after disposing the sample solution over the substrate.

Magnetic multi-bead assays including beads having magnetic properties that are related to each other have many advantages, such as improved sensitivity and specificity to target analytes.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1A schematically illustrates a functionalized superparamagnetic bead, and a functionalized ferromagnetic bead in accordance with one or more embodiments.

FIG. 1B schematically illustrates another functionalized superparamagnetic bead, and another functionalized ferromagnetic bead in accordance with one or more embodiments.

FIG. 1C schematically illustrates another functionalized ferromagnetic bead in accordance with one or more embodiments.

FIG. 2 schematically illustrates a complex that includes one of the functionalized superparamagnetic beads, a first moiety, one of the functionalized ferromagnetic beads, a second moiety, and an analyte in accordance with one or more embodiments.

FIG. 3 is a graph of Q(d_{A},d_{B}) as a function of the normalized ferromagnetic bead diameter d_{B}/d_{A} in accordance with one or more embodiments.

FIG. 4 illustrates a method of detecting a complex including an analyte in accordance with one or more embodiments.

FIG. 5 schematically illustrates a wide-field diamond magnetic imaging apparatus in accordance with one or more embodiments.

FIG. 6A schematically illustrates a complex that includes one of the functionalized superparamagnetic beads, one of the functionalized ferromagnetic beads, and an analyte in accordance with one or more embodiments.

FIG. 6B schematically illustrates a superparamagnetic bead, two ferromagnetic beads, and a complex including one superparamagnetic bead and one ferromagnetic bead in accordance with one or more embodiments.

FIG. 6C illustrates a positive magnetic image of the magnetic beads shown in FIG. 6B in accordance with one or more embodiments.

FIG. 6D illustrates a negative magnetic image of the magnetic beads shown in FIG. 6B in accordance with one or more embodiments.

FIG. 6E schematically illustrates one of the functionalized superparamagnetic beads and one of the functionalized ferromagnetic beads, in the absence of an analyte, in accordance with one or more embodiments.

FIG. 7 illustrates magnetic bead discrimination based on remanence and magnetic susceptibility in accordance with one or more embodiments.

### DETAILED DESCRIPTION

As stated above, various embodiments disclosed herein relate to methods and apparatus for detecting a complex including an analyte in a sample by observing complexes containing two or more distinguishable beads (i.e., beads having distinguishable magnetic properties) that are bound by the analyte. In various embodiments, magnetic multi-bead assays are used to determine analyte concentration in a sample by detecting magnetic fields produced by the two or more distinguishable beads. Detecting magnetic fields can include using any magnetic imaging technology, such as magnetic force microscopy, a scanning Hall probe, or a wide-field diamond magnetic imaging system, as described further below. Wide-field diamond magnetic imaging provides images of the vector magnetic field produced by a magnetic bead under a wide range of magnetic conditions. This general-purpose tool can be used to distinguish between magnetic bead types by, for example, measuring magnetic susceptibility and magnetic remanence at low applied field after first magnetizing the beads with a large magnetic field, as described further below.

### Superparamagnetic Bead A

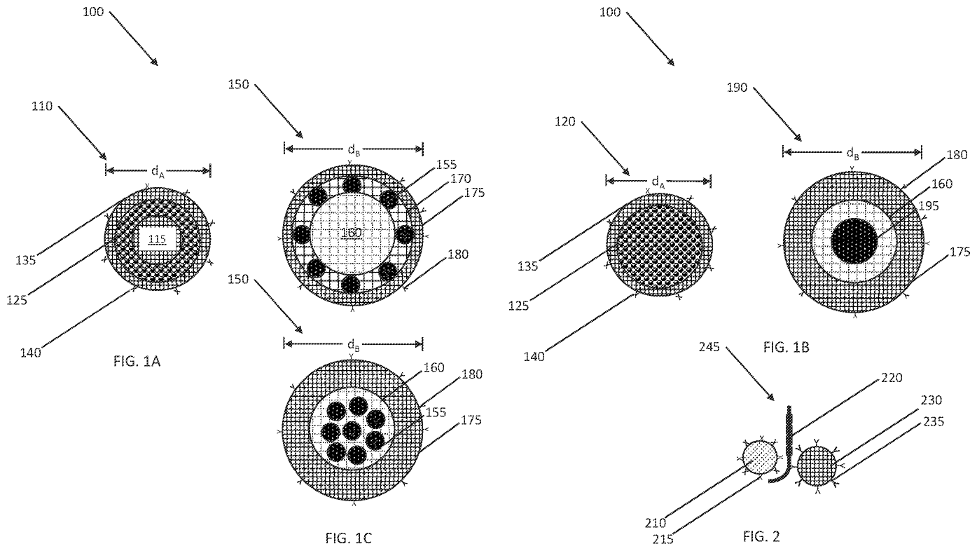

In accordance with one or more embodiments, as shown in FIGS. 1A and 1B, a bead system for bead-based analyte detection 100 includes a plurality of functionalized superparamagnetic beads A 110 and 120, one of each being shown in FIGS. 1A and 1B, respectively. The superparamagnetic beads A 110 and 120 each have a diameter d_{A} in a range of between about 0.1 μm and about 10 μm. The small diameter end of the range of 0.1 μm is set by the size of the magnetic nanoparticles 125 used to load them, which are typically in the range of between 5 nm and 20 nm. Examples of magnetically-loaded polymer beads smaller than 100 nm (0.1 μm) are difficult to find in the literature and, even if they could be synthesized, they would most likely have relatively inhomogeneous magnetic properties, because each bead would be loaded with a relatively small number of magnetic nanoparticles. Suitable superparamagnetic materials include, for example, iron oxide (Fe₂O₃ or Fe₃O₄), manganese ferrites (MnFe₂O₄), or cobalt ferrites (CoFe₂O₄), in the form of single-crystal nanoparticles less than about 20 nm in size, typically in a range of between 5 nm and 10 nm. The magnetic nanoparticles are engineered to be small enough that they exhibit a negligible amount of remanent magnetization in the absence of an applied field (i.e., superparamagnetism). When a magnetic field is applied, the particles magnetize in the direction of the field, producing a bead magnetization that is sufficient for magnetic separation.

The large diameter end of the range of 10 μm is set by imaging considerations in using magnetic imaging, such as a wide-field diamond magnetic imaging apparatus, described below, to avoid excessive bead crowding in the observed image. In a finite field of view, fewer large beads than small beads can be imaged. The diameter d_{A} can also be specified as having a peak of the size distribution that is less than 10 μm, as bead diameters are likely to be distributed over some range of sizes, or having half the beads in the sample have a diameter smaller than 10 μm. Considering the desirability of homogeneous magnetic properties at the low end, and imaging considerations at the high end, the diameter d_{A} is preferably in a range of between about 0.3 μm and about 3 μm, more preferably in a range of between about 0.5 μm and about 2 μm. As shown in FIG. 1A, the functionalized superparamagnetic bead 110 can include a nonmagnetic core 115 and superparamagnetic material 125 distributed substantially uniformly around the nonmagnetic core 115. Alternatively, as shown in FIG. 1B, the superparamagnetic material 125 can be distributed substantially uniformly throughout a volume of the functionalized superparamagnetic bead 120. As shown in FIGS. 1A and 1B, the functionalized superparamagnetic beads A 110 and 120 can optionally include a nonmagnetic buffer layer 135 around a surface of the bead 110 and 120, respectively. Suitable materials for the nonmagnetic buffer layer 135 and the nonmagnetic core 115 include polymers, such as polyethylene (PE), polytetrafluoroethylene (PTFE), and polymethylmethacrylate (PMMA), or other nonmagnetic materials. The nonmagnetic core 115 and the nonmagnetic buffer layer 135 each can be made of the same or different nonmagnetic materials.

The superparamagnetic beads A 110 and 120 each have a volume magnetic susceptibility X_{A} (dimensionless) that is in a range of between about 0.01 and about 10. The low end of 0.01 volume magnetic susceptibility is set by magnetic imaging requirements. As the volume susceptibility decreases, the magnetic moment of bead A becomes smaller (for a fixed bias magnetic field), and the signal becomes more difficult to detect. A bead with a volume magnetic susceptibility ten times smaller than the volume susceptibility of presently available beads would still be detectable. A further tenfold decrease in volume-susceptibility could be compensated for by increasing the bias magnetic field. The lower limit on the volume susceptibility is therefore chosen to be about a hundred times lower than the volume susceptibility of a typical example of presently available beads that are uniformly loaded throughout the full bead volume. (X_{A}=(81 m³/kg)·(1.7×10⁻⁵ g/cm³)=1.37, Dynabeads® MyOne, Thermo Fisher Scientific, Waltham, Mass.). See Fonnum, G. et al., “Characterisation of Dynabeads by magnetization measurements and Moessbauer spectroscopy”, J. Magn. Magn. Mater. 293, 41-47 (2005) that is incorporated by reference herein.

The high end of 10 on the superparamagnetic bead volume magnetic susceptibility is also set by magnetic imaging considerations. As the magnetic moment of bead A (which is produced by the bias magnetic field acting on the volume magnetic susceptibility of bead A) increases, the gradient of the magnetic field close to bead A becomes larger. As described further below, in wide-field diamond magnetic imaging, the fluorescence collected from a single imaging element of the diamond sensor is the sum over fluorescence emitted by all nitrogen-vacancy (NV) centers within the imaging element. If the gradient is large, different NV centers experience different magnetic fields and undergo different energy-level shifts, causing the optically detected magnetic resonance spectrum for that imaging element to blur. If the range of magnetic field values across a single imaging element is small, the average magnetic field in that imaging element can still be estimated. However, if the range of magnetic field values is large (i.e., substantially larger than 0.1 mT), the blurring becomes so great that the spectrum is poorly resolved, and therefore the sensitivity of the magnetic field measurement is reduced. The effect of a large bead A volume magnetic susceptibility can be countered to some extent by operating the sensor at a lower bias magnetic field, or by using a larger bead, but not by more than a factor of ten in either parameter. In practice, it is very unlikely that a bead with even ten times higher volume magnetic susceptibility can be synthesized, given that presently available beads (e.g., Dynabeads®) already contain about 10% magnetite nanoparticles by volume. The upper limit on volume magnetic susceptibility is therefore chosen to be about seven times higher than the volume magnetic susceptibility of a typical example (X_{A}=1.37×7=9.6≈10) of presently available beads. With the above magnetic imaging considerations, the magnetic susceptibility X_{A} is preferably in a range of between about 0.1 and about 5, more preferably in a range of between about 0.5 and about 3.

Turning back to FIGS. 1A and 1B, the superparamagnetic beads A 110 and 120 are functionalized to include a first moiety X 140 that associates with an analyte under suitable conditions in a sample solution. Only one of the first moiety X 140 is labeled in FIGS. 1A and 1B for clarity. Suitable moieties include a receptor, protein, antibody, cell, virus, or nucleic acid sequence.

### Ferromagnetic Bead B

In accordance with one or more embodiments, as shown in FIGS. 1A-1C, a bead system for bead-based analyte detection 100 also includes a plurality of functionalized ferromagnetic beads B 150 and 190 having a diameter d_{B} in a range of between about 0.1 μm and about 10 μm. The small diameter end of the range of 0.1 μm is set for similar reasons as described above for the functionalized superparamagnetic beads A 110 and 120, involving the size of the magnetic nanoparticles 155 used to load them, which are typically in the range of between 25 nm and 100 nm, as shown in FIGS. 1A and 1C. Only one of the magnetic nanoparticles 155 is labeled in FIGS. 1A and 1C for clarity. Suitable ferromagnetic materials include ferromagnetic cobalt ferrite nanoparticles 30 nm in size dispersed over the surface of a spherical polymer substrate 160 approximately 1 μm in diameter and adhered to the surface with an additional polymer layer 170, as shown in FIG. 1A. For homogeneous magnetic properties, the magnetic nanoparticles 155 are preferably uniformly dispersed around the spherical polymer substrate 160. Inhomogeneous magnetic properties are more undesirable for the ferromagnetic beads B than for the superparamagnetic beads A, potentially causing locally strong remanent magnetization in some beads, and requiring consideration of their total magnetic moment distribution, which might be expressed as a sum of multipole moments, including the dipole moment and higher order terms. For homogeneous magnetic properties, the functionalized ferromagnetic beads 190 can alternatively include ferromagnetic material 195 concentrated at a core of the spherical polymer substrate 160, as shown in FIG. 1B. In other embodiments, the ferromagnetic nanoparticles 155 can be distributed substantially uniformly throughout a volume of the spherical polymer substrate 160, as shown in FIG. 1C.

The large diameter end of the range of 10 μm is also set by magnetic imaging considerations of a wide-field diamond magnetic imaging apparatus, described below, to avoid excessive bead crowding on the diamond sensor. The diameter d_{B} can also be specified as having a peak of the size distribution that is less than 10 μm, as bead diameters are likely to be distributed over some range of sizes, or having half the beads in the sample have a diameter smaller than 10 μm. Considering the desirability of homogeneous magnetic properties at the low end, and imaging considerations at the high end, the diameter d_{B} is preferably in a range of between about 0.3 μm and about 3 μm, more preferably in a range of between about 0.5 μm and about 2 μm. As shown in FIGS. 1A-1C, the functionalized ferromagnetic beads 150 and 190 can optionally include a nonmagnetic buffer layer 175 around a surface of the bead 150 and 190, respectively. Suitable materials for the nonmagnetic buffer layer 175, the spherical polymer substrate 160, and the additional polymer layer 170 include polymers, such as polyethylene (PE), polytetrafluoroethylene (PTFE), and polymethylmethacrylate (PMMA), or other nonmagnetic materials. The spherical polymer substrate 160, the additional polymer layer 170, and the nonmagnetic buffer layer 175 each can be made of the same or different nonmagnetic materials. Since magnetic interactions weaken rapidly with increasing separation between beads, even a nonmagnetic layer 175 significantly thinner than the original bead radius can dramatically reduce dimer formation due to magnetic interactions, and therefore at least one of the functionalized superparamagnetic beads A 110 and 120 and the functionalized ferromagnetic beads 150 and 190 preferably includes a nonmagnetic buffer layer 135 or 175 around the surface of the bead.

The functionalized ferromagnetic beads 150 and 190 are functionalized to include a second moiety Y 180 that associates with the analyte under suitable conditions in the sample solution. Only one of the second moiety Y 180 is labeled in FIGS. 1A-1C for clarity. Suitable moieties include a receptor, protein, antibody, cell, virus, or nucleic acid sequence.

The ferromagnetic beads B 150 and 190 each have a magnetic dipole moment p_{B} that is in a range of between 0.02·(d_{B}/[μm])³ mA·μm² and

$${( \frac{20 {π}^{2} ( {k}_{B} T / [ J ] ) {( {d}_{A} / [ μ m ] )}^{3}}{( {μ}_{0} / [ \frac{J}{m {A}^{2} μ m} ] ) {X}_{A} Q} )}^{\frac{1}{2}} m A \cdot μ {m}^{2} ,$$

where μ₀ is the vacuum permeability, k_{B} is the Boltzmann constant, T is the temperature of the sample solution, and numerical values of Q(d_{A},d_{B}) are tabulated in Table 1.

The low end of the range of magnetic dipole moment p_{B} of 0.02·(d_{B}/[μm])³ mA·μm² is set by magnetic imaging requirements. The magnetic field produced at the diamond surface BB is proportional to

$${B}_{B} ∝ \frac{{p}_{B}}{{d}_{B}^{3}} \qquad ( 1 )$$

The characteristic values of these parameters typically used in a wide-field diamond magnetic imaging apparatus are p_{B}≈0.2-2 mA·μm² and d_{B}≈1 μm, resulting in a magnetic field BB as low as 1 μT, depending on the distance from the diamond surface and orientation of the bead B magnetization. A factor of ten signal reduction would likely be tolerable, but a lower signal would have a significantly negative impact on the assay and on the image acquisition time. The averaging time is expected to scale as the square of the signal, so even a tenfold reduction in signal can have a considerable impact on the viability of the assay. The lower limit of the magnetic dipole moment p_{B} of the functionalized ferromagnetic bead B is therefore chosen to be 0.02·(d_{B}/[μm])³ mA·μm², preferably 0.2·(d_{B}/[μm])³ mA·μm².

The high end of the range of magnetic dipole moment p_{B} is determined by the requirement that the non-specific magnetic interaction of the ferromagnetic bead B with the superparamagnetic bead A should be much weaker than the sample-specific antibody-mediated binding between the beads. In particular, the magnetic interaction energy Uᵢₙₜ between beads A and B when they are in physical contact must be less than or equal to five times the Boltzman energy at room temperature, 5k_{B}T, k_{B} being the Boltzmann constant and T being the temperature of the sample solution, typically room temperature. The upper limit on the magnetic interaction energy is related to typical values for the equilibrium constant K_{eq} of antibody reactions. As shown in FIG. 2, contact between a sample containing the analyte 220, such as an antigen, in the sample solution, the functionalized superparamagnetic beads 210 including a first moiety X 215, such as an antibody, that associates with the analyte 220 under suitable conditions, and the functionalized ferromagnetic beads 230 including a second moiety Y 235, such as another antibody, that associates with the analyte 220 under suitable conditions results in formation of a complex 245 including one of the functionalized superparamagnetic beads 210, the analyte 220, and one of the functionalized ferromagnetic beads 230. The analyte 220 is detected by co-localization of the functionalized superparamagnetic bead 210 and the functionalized ferromagnetic bead 230.

Using approximately equal (to within a factor of ten) concentrations of bead A, bead B, and antigen (i.e., analyte), to ensure that the number of nonspecific, magnetically bound A-B dimers is much less than the number of antigen-mediated A-B dimers in the magnetic imaging field of view, the equilibrium constant K_{eq}^{(AB)} of specific antigen-antibody reactions AB can be set to be 10⁶ times larger than the equilibrium constant for nonspecific magnetic interactions, denoted K_{eq}^{(mag)}. Given that the equilibrium constant ratio K_{eq}^{(AB)}/K_{eq}^{(mag)} is approximately proportional to the equilibrium concentration ratio [AB]/[mag] for the two types of dimers when the concentration of all components present is equal (such as in the imaging buffer immediately before the samples are placed on the diamond substrate for measurement), a “false positive” rate of approximately one in a million would be expected under these conditions. Magnetic field signatures are typically recorded from 1,000-10,000 magnetic objects (beads and bead complexes) in a single magnetic-imaging field of view, and therefore false positives are sure be avoided (i.e., less than one false positive) in the assay at this level, enabling a sensitive assay for K_{eq}^{(AB)}/K_{eq}^{(mag)}>10⁶. Typical values for the equilibrium constant K_{eq}^{(AB)} for antigen-antibody reactions are of the order of K_{eq}^{(AB)}=Kₐ^{(AB)}×C⁰≈10⁸-10¹⁰. Here, Kₐ^{(AB)} is the association constant for the antigen-antibody reaction, in units of M⁻¹, and C⁰=1 M is the standard state concentration. See Gilson, M. K. et al., “The Statistical-Thermodynamic Basis for Computation of Binding Affinities: A Critical Review”. Biophys. J. 74, 1047-1069 (1997), and Landry, J. P, Fei, Y., and Zhu, X., “Simultaneous Measurement of 10,000 Protein-Ligand Affinity Constants Using Microarray-Based Kinetic Constant Assays”. Assay Drug Dev. Technol. 10, 250-259 (2012), that are incorporated by reference herein.

To turn the ratio K_{eq}^{(AB)}/K_{eq}^{(mag)} of equilibrium constants into a limit on the magnetic interaction energy Uᵢₙₜ, note that the equilibrium constant can be related to the Gibbs free energy for a reaction according to

$$ln {K}_{e q} = - \frac{Δ G}{R T} . \qquad ( 2 )$$

Here, ΔG is the Gibbs energy (negative for an exothermal reaction), R is the molar Boltzmann constant R=8.31 J·mol⁻¹·K⁻¹, and T=293 K is the temperature of the sample solution, typically room temperature. See Gilson, M. K. et al. (Id.). Then, using the likely lower limit on the antigen-antibody reaction equilibrium constant of K_{eq}^{(AB)}>10⁸ cited above, the desired ratio of equilibrium constants yields

$$\begin{matrix}ln {K}_{e q}^{( m a g )} - ln {K}_{e q}^{( A B )} < ln {10}^{- 6} \\ \frac{- Δ {G}_{e q}^{( m a g )}}{R T} < ln {10}^{- 6} + ln {10}^{8} \\ Δ {G}_{e q}^{( m a g )} > - 4.6 R T\end{matrix} \qquad ( 3 )$$

Neglecting the decrease in entropy due to magnetic binding (which would only relax the constraint on the magnetic interaction if included, since ΔU^{(mag)}=ΔG^{(mag)}+TΔS^{(mag)}>ΔG^{(mag)}, for ΔS^{(mag)} negative), dropping the minus sign (since the magnetically bound state is understood to have lower energy), and expressing the last line in units of energy per bead pair (instead of per mole of bead pairs) yields Uᵢₙₜ<4.6 k_{B} T, or about Uᵢₙₜ≲5 k_{B} T, where k_{B} is the Boltzmann constant.

The magnetic interaction energy Uᵢₙₜ between beads A and B is related to the magnetic dipole moment p_{B} of bead B as described below. Assume that the ferromagnetic material is spatially concentrated in the ferromagnetic bead B, either at the surface of the polymer bead, as shown in FIG. 1A, or inside its volume, as shown in FIG. 1C, such that the ferromagnetic material can be approximated by a point dipole. Assume also that the superparamagnetic material is uniformly spatially distributed over the volume of the superparamagnetic bead A, as shown in FIG. 1B.

It has been empirically observed that a typical value of dipole moment for ferromagnetic beads is on the order of p_{B}≈1 mA·μm². This magnitude of dipole moment produces magnetic fields on the order of B_{typ}>0.1 T only at distances r<235 nm from the dipole because

$${B}_{t y p} ∼ \frac{{μ}_{0} {p}_{B}}{{r}^{3}} = \frac{[ 4 π \times 1 {0}^{- 4} T \frac{μ m}{m A} ] \cdot [ 1 mA μ {m}^{2} ]}{{r}^{3}} = \frac{1.3 \times 1 {0}^{- 3} T μ {m}^{3}}{{r}^{3}} , \qquad ( 4 )$$

and therefore r is approximately equal to

$$r ∼ {[ \frac{1.3 \times 1 {0}^{- 3} T μ {m}^{3}}{{B}_{t y p}} ]}^{\frac{1}{3}} = {[ \frac{1.3 \times 1 {0}^{- 3} T μ {m}^{3}}{0.1 T} ]}^{\frac{1}{3}} = 0.235 μ m . \qquad ( 5 )$$

Because the volume magnetic susceptibility of the superparamagnetic material only becomes appreciably nonlinear for applied fields B_{typ} greater than 0.1 T and hence distances r less than 235 nm, the volume fraction of a micrometer-scale superparamagnetic bead with a nonlinear response is small, even for a ferromagnetic dipole placed directly on the surface of the superparamagnetic bead. Therefore, a linear volume magnetic susceptibility is used for the superparamagnetic beads in the calculation below.

The total magnetic energy, Uᵢₙₜ, integrated over the volume of the superparamagnetic particle is

$${U}_{i n t} = \frac{1}{2} \int \vec{H} \cdot \vec{B} d V = \frac{{μ}_{0}}{2} \int {\vert \vec{H} \vert}^{2} ( 1 + {χ}_{A} ) dV . \qquad ( 6 )$$

The term containing the susceptibility describes the magnetic interaction energy; the term with 1 is just the self-energy of the dipole field in the absence of the superparamagnetic material. The maximum magnetic interaction energy, when the beads are in direct contact, is given by

$$\begin{matrix}{U}_{i n t} = \frac{{μ}_{0}}{2} \int {\vert \vec{H} \vert}^{2} {χ}_{A} {dV}^{′} \\ = \frac{{μ}_{0} {χ}_{A}}{2} \int {\vert \frac{1}{4 π} \frac{3 \vec{r} ( {\vec{p}}_{B} \cdot \vec{r} ) - {r}^{2} {\vec{p}}_{B}}{{r}^{5}} \vert}^{2} d {V}^{′} \\ = \frac{{μ}_{0} {χ}_{A} {p}_{B}^{2}}{2 {( 4 π )}^{2}} {\int }_{0}^{\frac{{d}_{A}}{2}} {r}^{′ 2} d {r}^{′ {\int }_{0}^{2 π} d {ϕ}^{′}} {\int }_{0}^{π} sin {θ}^{′} d {θ}^{′} [ \frac{3 {( \hat{p} \cdot \hat{r} )}^{2} + 1}{{r}^{6}} ] \\ = [ \frac{{μ}_{0} {χ}_{A} {p}_{B}^{2}}{4 {π}^{2} {d}_{A}^{3}} ] {\int }_{0}^{\frac{1}{2}} {ρ}^{′2} d {ρ}^{′} {\int }_{0}^{π} sin {θ}^{′} d {θ}^{′} [ \frac{3 {( \hat{p} \cdot \hat{ρ} )}^{2} + 1}{{ρ}^{6}} ] ] \\ = {U}_{0} Q .\end{matrix} \qquad ( 7 )$$

In the second-to-last step, the radial integration variables r′=ρ′·d_{A}/2 and r=ρ·d_{A}/2 were redefined to make the integral factor, Q, dimensionless. (Note, however, that Q is a function of the normalized bead B diameter, d_{B}/d_{A}.) The integral Q is numerically evaluated. Rewriting the limit on p_{B} in terms of the other bead parameters and Q yields:

$$\begin{matrix}{U}_{0} Q < {U}_{max} \\ [ \frac{{μ}_{0} {χ}_{A} {p}_{B}^{2}}{4 {π}^{2} {d}_{A}^{3}} ] Q < 5 {k}_{B} T \\ {p}_{B} < {( \frac{20 {π}^{2} ( {k}_{B} T / [ J ] ) {( {d}_{A} / [ μm ] )}^{3}}{( {μ}_{0} / [ \frac{J}{m {A}^{2} μ m} ] ) {X}_{A} Q} )}^{\frac{1}{2}}\end{matrix} \qquad ( 8 )$$

Equation 8 is the final statement of the upper limit on the magnetic moment p_{B} of the ferromagnetic bead, assuming it is mostly dipolar. Numerical values of Q(dA,dB) are provided in Table 1 over the entire range of ratios (0.01-100) of d_{B} and d_{A}, recalling that 0.1 μm<d_{A}<10 μm, and 0.1 μm<d_{B}<10 μm.

TABLE 1
Numerical Values of Q(d_{A,} d_{B})

| d_{B}/d_{A} | Q |
|---|---|
| 0.01 | 16016.83009 |
| 0.02 | 11997.09174 |
| 0.03 | 8962.381178 |
| 0.04 | 6626.082216 |
| 0.05 | 4870.304772 |
| 0.1 | 1160.371497 |
| 0.2 | 157.3417373 |
| 0.3 | 42.92606829 |
| 0.4 | 16.38938482 |
| 0.5 | 7.571908826 |
| 0.6 | 3.955713066 |
| 0.7 | 2.252355977 |
| 0.8 | 1.3670946 |
| 0.9 | 0.871853702 |
| 1 | 0.578453349 |
| 1.1 | 0.396446982 |
| 1.2 | 0.279190103 |
| 1.3 | 0.201214479 |
| 1.4 | 0.147941305 |
| 1.5 | 0.110686192 |
| 1.6 | 0.084096781 |
| 1.7 | 0.064775601 |
| 1.8 | 0.050509699 |
| 1.9 | 0.039824436 |
| 2 | 0.031717016 |
| 2.1 | 0.025492981 |
| 2.2 | 0.020663445 |
| 2.3 | 0.016879068 |
| 2.4 | 0.013886806 |
| 2.5 | 0.011501074 |
| 2.6 | 0.009584186 |
| 2.7 | 0.008032909 |
| 2.8 | 0.006769078 |
| 2.9 | 0.005732965 |
| 3 | 0.004878541 |
| 3.1 | 0.004170052 |
| 3.2 | 0.003579516 |
| 3.3 | 0.003084879 |
| 3.4 | 0.002668648 |
| 3.5 | 0.002316855 |
| 3.6 | 0.002018288 |
| 3.7 | 0.001763893 |
| 3.8 | 0.001546319 |
| 3.9 | 0.001359571 |
| 4 | 0.001198734 |
| 4.1 | 0.001059762 |
| 4.2 | 0.000939309 |
| 4.3 | 0.000834596 |
| 4.4 | 0.000743306 |
| 4.5 | 0.000663501 |
| 4.6 | 0.000593552 |
| 4.7 | 0.000532089 |
| 4.8 | 0.00047795 |
| 4.9 | 0.000430151 |
| 5 | 0.000387856 |
| 5.1 | 0.000350349 |
| 5.2 | 0.000317019 |
| 5.3 | 0.000287342 |
| 5.4 | 0.000260865 |
| 5.5 | 0.000237199 |
| 5.6 | 0.000216007 |
| 5.7 | 0.000196997 |
| 5.8 | 0.000179916 |
| 5.9 | 0.000164542 |
| 6 | 0.000150683 |
| 6.1 | 0.00013817 |
| 6.2 | 0.000126855 |
| 6.3 | 0.00011661 |
| 6.4 | 0.000107319 |
| 6.5 | 9.89E−05 |
| 6.6 | 9.12E−05 |
| 6.7 | 8.42E−05 |
| 6.8 | 7.79E−05 |
| 6.9 | 7.20E−05 |
| 7 | 6.67E−05 |
| 7.1 | 6.19E−05 |
| 7.2 | 5.74E−05 |
| 7.3 | 5.33E−05 |
| 7.4 | 4.96E−05 |
| 7.5 | 4.62E−05 |
| 7.6 | 4.30E−05 |
| 7.7 | 4.01E−05 |
| 7.8 | 3.74E−05 |
| 7.9 | 3.49E−05 |
| 8 | 3.26E−05 |
| 8.1 | 3.05E−05 |
| 8.2 | 2.85E−05 |
| 8.3 | 2.67E−05 |
| 8.4 | 2.51E−05 |
| 8.5 | 2.35E−05 |
| 8.6 | 2.21E−05 |
| 8.7 | 2.07E−05 |
| 8.8 | 1.95E−05 |
| 8.9 | 1.83E−05 |
| 9 | 1.72E−05 |
| 9.1 | 1.62E−05 |
| 9.2 | 1.53E−05 |
| 9.3 | 1.44E−05 |
| 9.4 | 1.36E−05 |
| 9.5 | 1.28E−05 |
| 9.6 | 1.21E−05 |
| 9.7 | 1.14E−05 |
| 9.8 | 1.08E−05 |
| 9.9 | 1.02E−05 |
| 10 | 9.68E−06 |
| 15 | 1.01E−06 |
| 20 | 1.97E−07 |
| 25 | 5.45E−08 |
| 30 | 1.89E−08 |
| 35 | 7.71E−09 |
| 40 | 3.53E−09 |
| 45 | 1.77E−09 |
| 50 | 9.53E−10 |
| 55 | 5.44E−10 |
| 60 | 3.25E−10 |
| 65 | 2.03E−10 |
| 70 | 1.31E−10 |
| 75 | 8.70E−11 |
| 80 | 5.94E−11 |
| 85 | 4.14E−11 |
| 90 | 2.95E−11 |
| 95 | 2.14E−11 |
| 100 | 1.58E−11 |

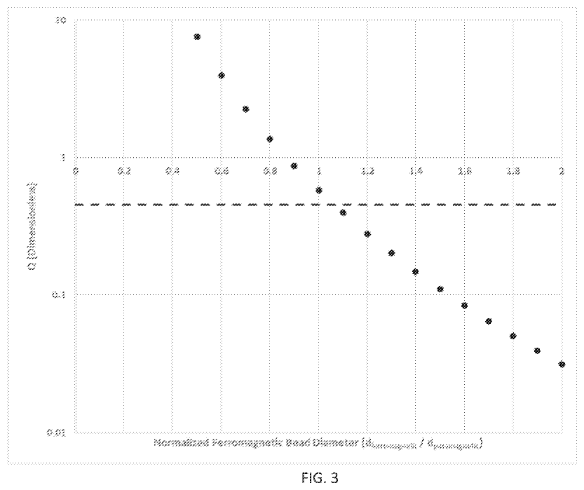

As a specific example, the dimensional pre-factor U₀ for the approximate bead parameters used in one assay, where d_{A}=1 μm, X_{A}=1.37, d_{B}=1.8 μm, and p_{B}=1.0 mA·μm² is calculated as shown below. The horizontal dashed line shown in FIG. 3 represents Uₘₐₓ/U₀ for these bead parameters.

$$\begin{matrix}{U}_{0} Q ( \frac{{d}_{B}}{{d}_{A}} ) = \vert \frac{{μ}_{0} {χ}_{A} {p}_{B}^{2}}{4 {π}^{2} {d}_{A}^{3}} ] \\ [ Q ( 1. 8 ) ] = [ \frac{( 4 π \times 1 {0}^{- 19} \frac{J}{m {A}^{2} μ m} ) \cdot ( 1.37 ) \cdot {( 1.0 m A μ {m}^{2} )}^{2}}{4 {{π}^{2} ( 1 μ m )}^{3}} ] \\ [ 0.05 ] = 2.2 \times 1 {0}^{- 21} J\end{matrix}$$

This value, 2.2×10⁻²¹ J, of the magnetic interaction energy Uᵢₙₜ is less than the allowed upper limit on the magnetic interaction energy, Uₘₐₓ=5 k_{B} T=5·[1.38×10⁻²³ J/K]·[293 K]=2.0×10⁻²⁰ J. Graphically, as shown in FIG. 3, the dots represent Q, a generic numerical function that depends only on the assumed particle geometries. The horizontal dashed line represents Uₘₐₓ/U₀, here equal to approximately 0.45, that can move up or down depending on d_{A}, X_{A}, and p_{B}. If the normalized diameter of bead B (=d_{B}/d_{A}), here equal to 1.8, is greater than the intersection of the two lines, here equal to approximately 1.0, then the condition is satisfied (1.8>1.0), and the non-specific magnetic interaction of the ferromagnetic bead B with the superparamagnetic bead A in the absence of the analyte will be substantially weaker than the analyte-specific antigen-mediated binding between the beads.

### Magnetic Multi-Bead Assay

The sensitivity of the magnetic multi-bead assay stems in part from three features:
(1) The assay measures co-presence of at least two distinguishable beads, such that detection of the target analyte only results from the analyte binding to at least two distinct antibodies on at least two distinguishable bead types, or binding to a polyclonal antibody on at least two distinguishable bead types, the polyclonal antibody binding to two different epitopes on the target antigen (i.e., target analyte). Having the analyte bound to at least two distinct antibodies provides enhanced target specificity through the combined specificity of multiple antibodies, which in turn provides better sensitivity.
(2) Confounding effects, such as signal backgrounds, caused by sample components other than the target analyte can be reduced or eliminated by purifying the sample using magnetic separation.
(3) Beads can be detected rapidly and with high accuracy and precision. Bead signals can be stronger and more stable and can be detected more quickly than signals from molecular reporters including fluorescent dyes and fluorescent products of enzymatic activity.

### Complex Formation

To measure a target analyte in a multi-bead assay, the analyte must bind to at least two distinguishable beads to form a complex, so that the presence of both beads can be detected. The beads can be coated with binding ligands, herein also denoted as moieties, such as antibodies, that bind specifically to and thereby associate with a certain region of a certain target analyte. Each bead in the multi-bead assay can be coated with one or more different types of binding ligands. Different bead types used in the multi-bead assay can have the same binding ligand types, overlapping sets of binding ligand types, or distinct binding ligand types. Two distinct bead types are used—herein denoted bead A and bead B. In the two-bead example, bead A and bead B are coated with antibodies, with antibody X on bead A and antibody Y on bead B. Antibody X binds specifically to a different region of the target analyte than antibody Y so that the target analyte can be bound to both simultaneously.

The complex, such as an immunocomplex, may be formed under suitable conditions, such as by incubating the sample solution with a suspension of bead A and bead B. Target analytes in the sample will encounter a bead surface as they diffuse through the sample, and bind to it. The sample solution can be mixed, shaken, or otherwise agitated to accelerate this process. As the beads also move through the sample solution, they will encounter analytes bound to beads of the opposite type and will additionally bind to those analytes, forming heterogeneous bead complexes of the form A-B, A-B-A, B-A-B, and other combinations.

The beads and bead complexes are then concentrated together by magnetic separation. First, a magnetic field gradient is applied that exerts a magnetic force on superparamagnetic bead A and ferromagnetic bead B. Any bead A, bead B, and bead complex containing bead A or bead B will be separated from the sample, forming a “pellet” of magnetic material and bound analytes. Unbound sample components, referred to here as “background material,” will not be separated and therefore can be discarded with the supernatant above the pellet. The magnetic field gradient can then be removed and the beads can be re-suspended in the same or different buffer solution. This process can be repeated to reduce the concentration of background material. Magnetic separation can be performed by hand or automated with a commercial plate washer.

Alternately, complex formation can also be performed sequentially in discrete steps, which can reduce signal background caused by nonspecific binding of beads into complexes in the absence of the target analyte. First, bead A can be added to the sample to capture the target analyte, followed by applying a magnetic field gradient to reduce the concentration of background material by magnetic separation. Bead B can then be added separately to this purified sample. Whether bead A and bead B are added together simultaneously or sequentially will be determined empirically and will depend on the antibodies utilized and on whether nonspecific binding is significantly reduced using sequential binding steps.

If bead B is less magnetic than bead A, then magnetic separation can be used after forming complexes to reduce signal background associated with unbound bead B. For example, a less-magnetic bead B will be separated from the sample suspension more slowly than bead A, so that magnetic separation can be terminated at a point at which bead A has been suitably separated into a pellet while the separation of bead B remains incomplete. If at this point the supernatant above the pellet is discarded, a significant fraction of bead B will be removed, but bead A will be preserved, including complexes containing bead A.

As described above, the diameters of bead A and bead B can be in a range of between 0.1 μm and 10 μm. Bead A and bead B can be chosen to have different diameters, such that the diameter of the functionalized superparamagnetic beads is different from the diameter of the functionalized ferromagnetic beads by at least 50%, so that the two bead types may be distinguished by the spatial distribution of their respective magnetic field signals. Alternatively bead A and bead B can be chosen to have similar diameters, that is, diameters different by less than 50%, so that they exhibit similar surface area, move similarly in the liquid sample suspension, occupy a similar amount of space in the detection region, and provide similar signal magnitudes. Bead diameters in the range of 0.5 μm to 5 μm can facilitate rapid magnetic separation (in a matter of seconds) and provide substantial surface area for a large quantity of binding ligands on each bead. In addition, bead diameters in this range are similar to or slightly larger than the typical diffraction-limited imaging resolution of an optical microscope or a wide-field diamond magnetic imaging system.

### Complex Detection

Once complexes, such as immunocomplexes, have been formed (heterogeneous bead complexes containing bead A and bead B bound by the analyte), they are measured by detecting the co-presence of both bead types as described further below. The measurement of complexes containing the analyte can be calibrated with a range of calibration samples of known analyte concentration so that a given measurement of complexes implies a certain analyte concentration. The measurements of the range of calibration samples is collectively referred to as a calibration curve. Detection of complexes, by the methods and apparatus described herein, enables measuring analyte concentration in combination with a calibration curve.

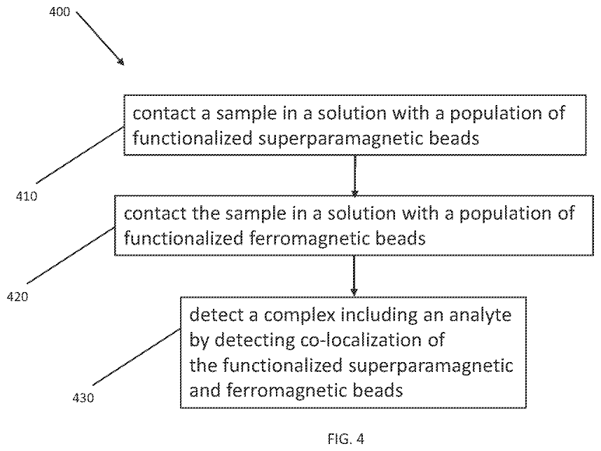

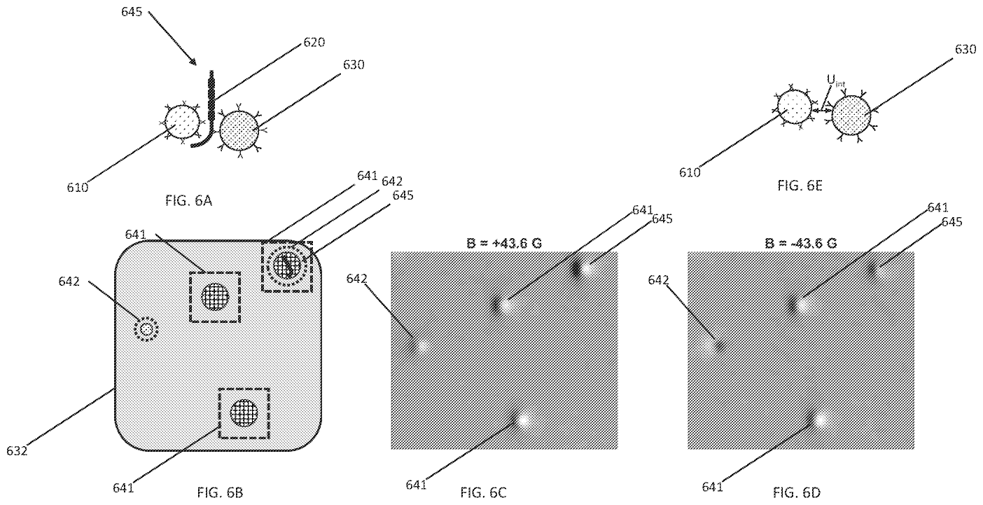

In accordance with one or more embodiments, as shown in FIG. 4, a method 400 of detecting a complex including an analyte includes contacting 410 a sample solution potentially containing an analyte with a population of functionalized superparamagnetic beads, which are functionalized to include a first moiety that associates with the analyte under suitable conditions, and contacting 420 the sample solution with a population of functionalized ferromagnetic beads, which are functionalized to include a second moiety that associates with the analyte under suitable conditions. The order of the contacting steps 410 and 420 can also be reversed. Contact results in formation of complexes, each including one of the functionalized superparamagnetic beads, the analyte, and one of the functionalized ferromagnetic beads. Contact in the absence of the analyte in the sample solution, as shown in FIG. 6E, results in a magnetic interaction energy Uᵢₙₜ between the functionalized superparamagnetic bead A 610 and the functionalized ferromagnetic bead B 630, the magnetic interaction energy Uᵢₙₜ being less than or equal to 5k_{B}T, k_{B} being the Boltzmann constant and T being the temperature of the sample solution. Turning back to FIG. 4, the method further includes detecting 430 the complex including the analyte by detecting co-localization of the functionalized superparamagnetic bead and the functionalized ferromagnetic bead. In some embodiments, the method can further include applying a magnetic field gradient to the sample solution after contacting the sample with the population of functionalized superparamagnetic beads. In other embodiments, applying the magnetic field gradient to the sample solution can be performed after contacting the sample solution with the population of functionalized ferromagnetic beads. In some embodiments, the population of functionalized superparamagnetic beads and the population of functionalized ferromagnetic beads can be added to the sample solution sequentially. In other embodiments, the population of functionalized superparamagnetic beads and the population of functionalized ferromagnetic beads can be added to the sample solution simultaneously. In some embodiments, the method can further include applying a magnetic field gradient to the sample solution after contacting the sample with the population of functionalized superparamagnetic beads. In other embodiments, applying the magnetic field gradient to the sample solution can be performed after contacting the sample solution with the population of functionalized ferromagnetic beads. In some embodiments, the population of functionalized superparamagnetic beads and the population of functionalized ferromagnetic beads can be added to the sample solution sequentially. In other embodiments, the population of functionalized superparamagnetic beads and the population of functionalized ferromagnetic beads can be added to the sample solution simultaneously. In some embodiments, detecting co-localization of the functionalized superparamagnetic bead and the functionalized ferromagnetic bead includes detecting magnetic fields using any magnetic imaging technology, such as magnetic force microscopy, a scanning Hall probe, or a wide-field diamond magnetic imaging system. Wide-field diamond magnetic imaging with nitrogen-vacancy (NV) centers in diamond is capable of rapidly imaging magnetic fields disposed over the surface of a diamond sensor, at room temperature, with sub-micron resolution.

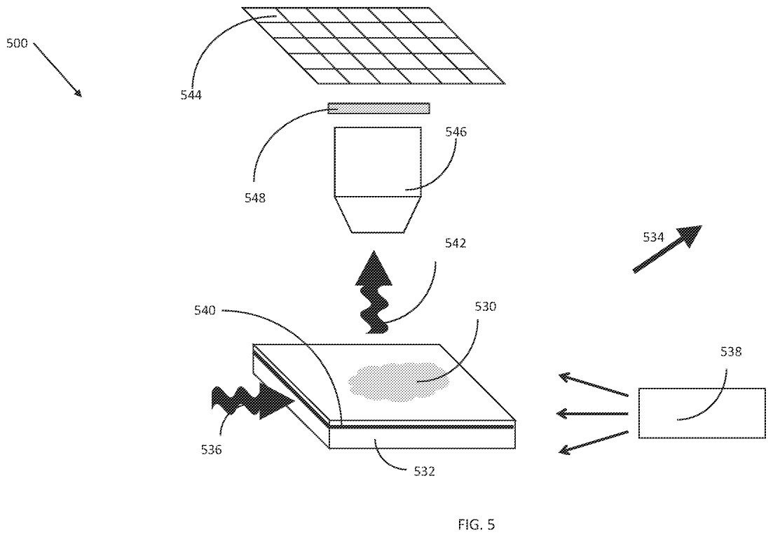

In accordance with one or more embodiments, as shown in FIG. 5, a system, such as a wide-field diamond magnetic imaging system 500 for detecting a complex including an analyte based on optically detected magnetic resonance (ODMR) includes a substrate 532 including at least one ODMR center 540 (a plurality of ODMR centers 540 shown in FIG. 5), a light source 536 configured to generate incident light that excites electrons within the at least one ODMR center 540 from a ground state to an excited state, a magnet 534 for applying a bias magnetic field on a sample solution 530 disposed over the at least one ODMR center 540, the sample solution 530 including complexes 245, each including one of the functionalized superparamagnetic beads 210, the analyte 220, and one of the functionalized ferromagnetic beads 230 shown in FIG. 2. The ODMR center 540 is a localized crystallographic defect having a spin structure that can be measured by ODMR. The sample solution 530 need not be in direct contact with the ODMR center 540. Further details of sampling configurations are described in PCT Patent Application No. PCT/US2018/044409 filed on Jul. 30, 2018 and entitled METHODS AND APPARATUS FOR SAMPLE MEASUREMENT that is incorporated by reference herein. Turning back to FIG. 5, the wide-field diamond magnetic imaging system 500 further includes a microwave source 538 configured to generate a microwave field incident on the at least one ODMR center 540, the microwave source 538 being further configured to generate the microwave field with frequencies that correspond to ground state transitions in the at least one ODMR center 540, in which the at least one ODMR center 540 produces emitted light 542 when illuminated by the incident light 536, characteristics of the emitted light 542 being influenced by the microwave field and by the functionalized superparamagnetic bead 210 and the functionalized ferromagnetic bead 230 associated with the analyte 220 in the sample solution 530. In the embodiment shown in FIG. 5, the plurality of ODMR centers 540 are nitrogen-vacancy (NV) centers in a diamond lattice, formed in an upper surface of the diamond substrate 532. In another aspect, the plurality of ODMR centers can be silicon-vacancy centers in a silicon carbide lattice, or in a diamond lattice. Turning back to FIG. 5, under optical excitation 536, fluorescence 542 emitted from a thin layer of ODMR centers 540 near the surface of the diamond substrate 532 is imaged onto an optical photodetector array 544, that is an optical imaging system having an imaging sensor such as a charge-coupled device (CCD) or complementary metal oxide semiconductor (CMOS) camera. The variation of ODMR center fluorescence under microwave excitation reveals the ODMR electron spin resonance (ESR) frequency, and hence the magnetic field shift of the ODMR spin sublevels. The spatial structure of the magnetic field at the diamond surface created by the sample (i.e., complex) 530 can thus be determined from images of ODMR center fluorescence 542, whose characteristics are influenced by the microwave field and by the magnetic field created by the superparamagnetic bead 210 and the functionalized ferromagnetic bead 230 associated with the analyte 220 in the complex 530.

Briefly, the process to acquire a magnetic image is as follows:
1. Dispose a magnetic sample (i.e., complex) 530 to be imaged over, onto, or near to the sensing surface 540 of the diamond substrate 532. An intermediate layer (not shown) may be interposed between the sample 530 and the diamond substrate 532.
2. Apply a magnetic bias field 534 in an arbitrary direction.
3. Illuminate the ODMR centers 540 in the diamond center with green light 536 (near 532 nm wavelength).
4. Apply a microwave field from a source 538 to the diamond, with frequency near one of the ODMR center ESR transitions.
5. Acquire an image of ODMR center fluorescence 542 emitted from the sensing surface 540 at optical detector array 544 through imaging objective 546 and optical filter 548.
6. Repeat steps 4-5 using different microwave frequencies that span one or more ranges around one or more NV center ESR transitions. The result is a stack of images, each corresponding to a different microwave frequency.
7. Repeat steps 4-6 one or more times, averaging the results to reduce imaging noise in the image stack.
8. For each image pixel in the image stack, construct an ESR spectrum from that pixel's value across all images in the stack. Analyze this spectrum to determine the frequencies of one or more ESR transitions.
9. For each image pixel in the image stack, compute the magnetic field based on the frequencies of observed ESR transitions at that pixel.

The applied magnetic field 534 induces magnetization in bead A and an associated magnetic field from the bead. A magnetic field in the range of 0.5 to 10 mT, which may be generated with permanent magnets or an electromagnet, is sufficient to resolve features in the electron spin resonance spectrum of the diamond imaging sensor. The diamond magnetic imager images these bead fields directly, allowing for individual bead detection and location.

Additionally, magnetic imaging is particularly insensitive to signal backgrounds due to unwanted light, detector noise, and sample contaminants that fluoresce, scatter, or absorb light. Magnetic signal backgrounds are extremely low in biological samples and they do not impede the ability to measure even modestly magnetic beads.

Distinguishing Magnetic Bead Types with Magnetic Imaging

Wide-field diamond magnetic imaging directly images the vector magnetic field produced by a magnetic bead under a wide range of magnetic conditions. This general-purpose tool may be used to distinguish between magnetic bead types over a wide range of different properties.

In accordance with one or more embodiments, as shown in FIG. 6A, bead A 610 is superparamagnetic and bead B 630 is ferromagnetic, substantially similar to the functionalized superparamagnetic beads A and functionalized ferromagnetic beads B shown in FIG. 2 and described above. Magnetic imaging with single-bead spatial resolution is used to identify bead A 610, and also to identify bead B 630, distinguishing between the two. In one embodiment, bead A 610 and bead B 630 are distinguished by measuring magnetic susceptibility and magnetic remanence at low applied field after first magnetizing the beads with a large magnetic field. For example, bead A 610 contains a quantity of iron oxide such that the magnitude of the average induced magnetization of bead A 610 with an applied bias field of 4 mT is approximately 3×10⁻¹⁵ A m². Bead B 630 has a remanent magnetization fraction of greater than 50%, such that, after being magnetized in a field of at least 300 mT and once the magnetizing field has been removed, bead B 630 retains a large proportion of its saturated magnetization value. For example, bead B 630 contains a quantity of cobalt ferrite such that the magnitude of the average remanent magnetization of bead B 630 after the magnetizing field is removed is approximately 2×10⁻¹⁵ A m².

A magnetic imaging procedure is described below for identifying complexes 645 containing the target analyte 620, bead A 610 and bead B 630. After forming complexes 645 in a sample suspension, a representative portion of the sample is disposed over and dried on the surface 632, shown in FIG. 6B, of a diamond magnetic imaging sensor shown in FIG. 5. The sensor's imaging surface is a {100} face and this surface contains a thin layer approximately 1-μm thick that is rich in nitrogen-vacancy (NV) centers. Turning back to FIGS. 6B-6D, after magnetic imaging, complexes 645 are identified by identifying bead A 610 and bead B 630 in close proximity to one another, including close enough to be spatially unresolved in the images. Prior to magnetic imaging, a magnetizing field is applied in a direction normal to the horizontal diamond surface. A field of greater than 200 mT applied for a period of several seconds is sufficient to magnetize the magnetic material in bead A. The dried sample is then magnetically imaged twice with a bias magnetic field of 4 mT applied parallel to one crystal axis of the diamond sensor, which is oriented at an angle of approximately 35 degrees with respect to the imaging surface. The 4 mT imaging field is reversed between acquiring the two magnetic images, shown in FIGS. 6C and 6D, termed the positive (FIG. 6C) and negative (FIG. 6D) images, denoting the +4 mT and −4 mT imaging fields, respectively. The magnetic images measure the projection of the sample magnetic field vector onto the axis of the imaging field.

Given that bead A 610 is superparamagnetic, the greater than 200 mT magnetizing field does not leave bead A 610 with significant remanent magnetization. In both the positive and negative images, the magnetization of bead A is only that magnetization which is induced in the superparamagnetic beads by the 4 mT imaging field. Bead A 610 produces the same feature 641 in both magnetic images, since the bead A 610 magnetization vector is in both cases parallel to the imaging field.

In contrast, the greater than 200 mT magnetizing field leaves bead B 630 strongly magnetized in the vertical direction, oriented up with respect to the horizontal diamond sensor imaging surface. Once the magnetizing field is removed, the weaker 4 mT imaging field does not significantly change the magnetization of bead B 630, since the magnetic susceptibility of bead B 630 near zero magnetic field, when previously magnetized along the same axis, is low. Therefore, bead B 630 produces an image feature 642 that inverts sign between the positive and negative magnetic images, with positive magnetic field projection changing to negative and vice versa, as illustrated in FIGS. 6B, 6C, and 6D.

All magnetic objects identified in the magnetic image field of view are quantified by magnetization, such that bead A 610 is assigned a positive value in both images and bead B 630 is assigned a positive and negative value in the positive and negative images, respectively. Bead complexes 645 will be assigned magnetization values that reflect the complex composition. For example, bead dimers of the form A-A or B-B will generally be assigned larger values with the same sign of bead A or bead B monomers, respectively. Bead dimers 645 of the form A-B or larger heterogeneous bead complexes will be assigned values of smaller magnitude in the negative image (FIG. 6D) than in the positive image (FIG. 6C), reflecting oppositely-magnetized beads within the complex.

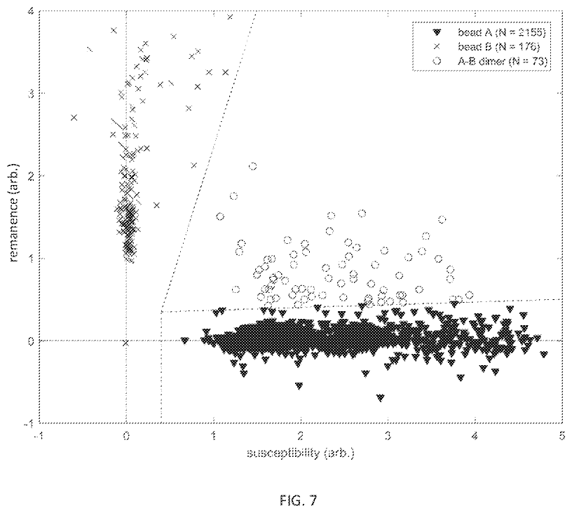

All magnetic objects in the magnetic images may be represented on a scatter plot whose axes are the sum and difference, respectively, of the positive and negative image magnetization values. This sum and difference may also be termed the susceptibility and remanence of the single-bead magnetization curve, as they are approximately proportional to these properties. As shown in FIG. 7, bead A and bead complexes containing only bead A will be clustered near one axis, with large susceptibility and zero remanence, 2,155 counts being shown in FIG. 7; bead B and bead complexes containing only bead B will be clustered near the other axis, with large remanence and near-zero susceptibility, 176 counts being shown in FIG. 7. Complexes containing both bead A and bead B will exhibit significant susceptibility and remanence, so they may be identified as the objects in the scatter plot in a region sufficiently separated from both axes, 73 counts being shown in FIG. 7. This region is unlikely to contain signals from bead A or bead B alone, or from homogeneous bead complexes such as those of the form A-A or B-B.

### Accelerated Bead Interaction Kinetics

It is known in the art that immunoassays must allow time for target analytes in a liquid sample to bind to antibodies that enable detection of the target analytes. Depending on the reagent concentration and sample conditions (such as temperature, viscosity, and process for agitating or mixing the sample), several minutes may be required for most analytes to become bound, even when there is a large excess of binding sites available, due to the time needed for the analyte to move through the sample by diffusion or active shaking or stirring.

The rate of interactions between different beads in the sample suspension may determine multi-bead assay speed, since bead diffusion is generally slower than diffusion of smaller molecular analytes. Since a bead-bound target analyte may also occupy a relatively small fraction of the bead's surface area, when the bead to which the analyte is bound interacts with a second bead, the analyte may not be exposed to the second bead in a manner conducive for binding (e.g., the interaction occurs on the side of the first bead opposite to where the analyte is located). Several bead interactions may be required on average to form an immunocomplex. The multi-bead assay time can be shortened by concentrating the sample solution after contacting the sample solution with the population of functionalized ferromagnetic beads to induce bead-bead interactions that lead to immunocomplex formation, accelerating bead kinetics beyond what may be expected for diffusion or stirring alone.

In one embodiment, bead-bead interactions may be induced by agglomerating a plurality of functionalized superparamagnetic and ferromagnetic beads, after contacting the sample solution with the population of functionalized ferromagnetic beads, before detecting the complex, by means of spinning the sample suspension on a centrifuge to concentrate the beads into a pellet in the sample tube. This process can be performed in less than a minute with standard benchtop centrifuge systems. Beads in the pellet may be closely spaced or in contact with one another, resulting in many bead interactions in the pellet. The pellet can be re-suspended by mixing the suspension. This centrifuge process can be repeated as necessary to ensure sufficient interactions between beads to form immunocomplexes containing the target analyte.

In another embodiment, magnetic separation can be used to form a pellet of beads in the sample suspension and induce bead-bead interactions by applying a magnetic field gradient to the sample solution after contacting the sample solution with the functionalized superparamagnetic and ferromagnetic beads. As with the centrifuge process, this magnetic approach to accelerating bead kinetics can be performed in less than a minute and repeated as necessary to form immunocomplexes containing the target analyte. The magnetic approach can be performed with permanent magnets for a simple, inexpensive, and compact process with minimal power consumption. An electromagnet can also be used to apply the magnetic field with no moving parts.

A bead pellet produced by magnetic separation of a sample suspension can be agitated without removing the magnetic field, by varying or otherwise changing the magnetic field gradient applied to the sample solution with respect to the beads. For example, the field magnitude, direction, or spatial distribution can be changed or oscillated to apply different magnetic forces on the beads. Alternatively, or additionally, the sample tube can be moved with respect to the magnetic field. For example, rotating the tube can move the pellet away from its equilibrium position so that the pellet will be dragged by the magnetic field gradient to a new position. These changes will cause beads in the pellet to move with respect to each other and can induce additional bead interactions and immunocomplex formation.

In yet another embodiment, a permanent magnet can be moved relative to a tube containing a sample suspension with magnetic beads of multiple types. The magnet can follow a fixed pattern of motion. Examples include the magnet orbiting the sample tube in a circle, rotating on its own axis, or rocking back and forth between two points. The motion can be continuous, in which case the bead pellet will continuously move through the tube, subjecting the beads to shear forces from the liquid, tube walls, and other beads. This motion and the associated forces on the beads will agitate the pellet continuously to drive bead-bead interactions. In another example, the motion can occur in discrete periods separated by periods of rest, in which the bead pellet concentrates to a higher bead density than is achieved during continuous motion. If the different bead types respond significantly differently to the field of the permanent magnet, then the periods of rest will enable the multiple bead types to co-localize more effectively than during continuous motion.

In still another embodiment, a plurality of permanent magnets can be moved relative to a plurality of samples in separate wells of a plate, such that the sample in each well is subjected to a magnetic field profile in time and space that is substantially similar. This approach enables driving bead-bead interactions in parallel over a plurality of samples for improved sample preparation throughput.

Accelerating bead kinetics and the rate of bead-bead interactions in sample suspension decreases the time required to bind target analytes into detectable multi-bead complexes, yielding a faster assay.

This method can also enable the use of a lower quantity of binding ligands on the bead surfaces, because the likelihood of a given ligand to bind to the target analyte is increased by the increased frequency of bead-bead interactions. Using fewer binding ligands can reduce the cost of the assay significantly.

### Multiplexing

It is often useful for an assay to measure the concentration of multiple distinct analytes in a single sample. A multiplexed assay measures distinct target analytes by associating a distinct signal with each target, so that the analyte signals can be distinguished in the assay measurement. The magnetic dual-bead assay can be generalized to a multiplexed multi-bead assay by using more than two distinguishable bead types. Different analytes may be specifically detected by observing the formation of analyte-specific complexes including a plurality of functionalized beads of at least a third type, functionalized to include at least a third moiety that can specifically associate with at least a second analyte under appropriate conditions.

Distinct magnetic bead types can be distinguished by preparing bead types with different magnetic properties that can be distinguished by magnetic imaging. In an exemplary case, the distinct bead types can be prepared by loading each bead type with specific and distinguishable quantities of magnetic material. Alternatively, distinct bead types can be prepared by loading each bead type with different magnetic material exhibiting different properties. The fully magnetic multi-bead assay described above discriminates between two bead types in this manner. A multiplexed assay can be implemented by adding additional distinguishable beads using distinct combinations of the properties described above. For example, bead A may be superparamagnetic, while bead B and C are ferromagnetic with different coercivities derived from bead B and bead C comprising different ferromagnetic materials. In this case, the beads can be distinguished by measuring magnetic remanence after magnetization, and then also measuring whether this remanence is reversed upon application of a demagnetizing field that exceeds the coercivity of bead B, but not that of bead C. The three beads in this case can be used to implement a multiplexed assay for three analytes using an embodiment described above.

### Sample Preparation

One example of suitable conditions for sample preparation for the multi-bead assay is to combine a few drops of blood with the multi-bead mixture, incubate for a few minutes with accelerated kinetic mixing and deposit the sample solution on the diamond surface to dry followed by magnetic imaging. The sample solution can be partially or completely dehydrated before detecting the complex.

A suitable sample preparation proceeds as follows: plasma or serum is diluted in assay buffer 1 to 100 fold (e.g., 10-fold by adding 5 μL sample into 45 μL) assay buffer and briefly vortex mixed. This diluted sample is further diluted 2× with 50 μL of bead mix for a final volume of 100 μL. The bead mix includes 2000-1,000,000 superparamagnetic beads (e.g., 100,000 beads A) and ˜2000-1,000,000 ferromagnetic beads (e.g., 100,000 beads B). The final assay reaction is a 2-200-fold dilution (e.g., 20 fold) of sample in 100 μL. The assay reaction is incubated with vortex mixing at 50-1200 rpm (e.g., 800 rpm) for 15 minutes at room temperature. The samples are then placed in a centrifuge and spun at 500-10,000 g (e.g., 1500 g) for 0.5-30 minutes (e.g., 3 minutes), followed by pulse vortex mixing. The centrifugation and mixing cycle are repeated from zero to 5 more times (e.g., twice more), after which the sample is placed against a permanent magnet (magnetic field ˜300 mT) for 5-300 seconds (e.g., 30 seconds) to pellet the magnetic beads against the sidewall of the reaction tube. The assay volume is removed by pipette leaving the bead pellet intact on the side wall against the magnet. The tube is removed from the magnet and the pellet is suspended in 50-1000 μL (e.g., 500 μL) of wash buffer by vortex mixing. The tube is pulse spun at 500-20,000 g (e.g., 1500 g) for 1-5 seconds (e.g., 3 seconds) to remove fluid from the cap, and placed on the magnet for 10-300 seconds (e.g., 30 seconds). The wash cycle is repeated 0-5 (e.g., 2) more times for a total of 1-6 (e.g., 3) washes. The pellet is washed one time with 200 μL of imaging buffer and finally suspended in ˜40 μL of imaging buffer. About 10 μL is applied to the diamond sensor for magnetic imaging.

An alternative sample preparation proceeds as follows: plasma or serum is diluted in assay buffer 1-100 fold (e.g., 10-fold by adding 5 μL sample into 45 μL) into assay buffer and briefly vortex mixed. This diluted sample is placed in an assay plate (e.g., 96 well) further diluted 2× with 50 μL of bead mix for a final volume of 100 μL. The bead mix includes about 2,000-1,000,000 (e.g., about 300,000 beads A) superparamagnetic beads and about 2000-1,000,000 (e.g., 100,000 beads B) ferromagnetic beads. The final assay reaction is a 1-100 fold (e.g., 20-fold) dilution of sample in 100 μL. The assay reaction is incubated with vortex mixing (e.g., 850 rpm) for 15 minutes at room temperature on a plate mixer. The samples are then placed in a plate centrifuge and spun at 100-20,000 g (e.g., 850 g) for 10-300 seconds (e.g., 2 minutes), followed by pulse vortex mixing. The centrifugation and mixing cycle are repeated (e.g., once more), after which the sample is placed against a permanent magnet (magnetic field about 300 mT) for about 5-300 seconds (e.g., about 60 seconds) to pellet the magnetic beads against the sidewall of the reaction tube. The assay volume is removed by pipette leaving the bead pellet intact on the side wall against the magnet. The plate is removed from the magnet and the pellet is suspended in 50-400 μL (e.g., 250 μL) of wash buffer by vortex mixing. The plate is placed on the magnet for about 5-300 seconds (e.g., about 60 seconds). The wash cycle is repeated 1-5 more times (e.g., once) for a total of 2-6 (e.g., 2) washes. The pellet is finally suspended in about 5-100 μL (e.g., 40 μL) of imaging buffer. About 10 μL is applied to the diamond sensor (or thin membrane cartridge) for magnetic imaging.

The sample can be any chemical or biological sample, such as whole blood, blood components (plasma, serum), tissue culture, cell culture, bodily fluids (cerebral spinal fluid (CSF), tears, saliva, breast milk, urine, semen, nasal discharge), tissue samples (oral swabs, biopsies, surgical resections), recombinant DNA, RNA or protein, endogenous DNA, RNA or protein, synthetic nucleic acids or protein peptides.

Further sample requirements may include volumes of sample types from 0.1 μL to 1000 μL, and dilution of sample types and volume into assay buffers. Dilutions of sample types may include dilution by a factor of 2-1,000. Assay buffers may be determined empirically for optimized signal generation and minimized non-specific background, or false binding of any kind.

Samples can be combined in various ways including, for example, with multi-bead mixtures in blood collection tubes, assay tubes, assay plates/well, microfluidic devices, reaction chambers, incubation chambers, lateral flow devices, blood component separation devices, or other liquid handling or manipulation devices.

Samples can be mixed in various ways including, for example, by magnetic fields, centrifugal force, gravity, sound induced, light induced, electric induced, ionic interactions, van der Waals induced, Brownian motion, spinning, or other mechanical means.

Samples can be mixed with multi-bead mixtures for times necessary to capture targets of interest ranging from, for example, a second to several hours.

Samples need not be washed or processed beyond dilution with bead mixture, or washed by manual pipette or on a plate washer or liquid handler. Washed samples can use a buffer composition empirically determined for an assay or use salt concentrations from 0.1× to 10× (i.e. PBS) or from 1 mM to 4 M NaCl and/or other biological salt solution. Wash volumes can be from 0-400 μL in assay plates or from 0-2.0 ml in assay centrifuge tubes.

Samples can be introduced to the magnetic imaging device in various ways including, for example, by pipette, capillary flow tube or device, sample handling device, liquid handling device, integrated device, lateral flow device, disposable or reusable device.

Samples can be deposited on the diamond surface by several modes of application including, for example, pipetting, pouring, dripping, capillary flow, pumping, gravity induced flow, magnetic induced flow, ionic induced flow, sound induced flow, light induced flow, mechanical vibration induced flow, sheath flow, centrifuge induced, and thermal induced flow.

Samples can be magnetically imaged in a dry, dehydrated (i.e., partially dry or gel), or wet state.

### Further Example Embodiments

Example 1 is a bead system for bead-based analyte detection that includes a plurality of functionalized superparamagnetic beads having a diameter d_{A} and a volume magnetic susceptibility X_{A}. The functionalized superparamagnetic beads are functionalized to include a first moiety that associates with an analyte under suitable conditions in a sample solution. The system further includes a plurality of functionalized ferromagnetic beads having a diameter d_{B} and a magnetic dipole moment p_{B}. The functionalized ferromagnetic beads are functionalized to include a second moiety that associates with the analyte under suitable conditions in the sample solution. Contact between a sample containing the analyte in the sample solution, the functionalized superparamagnetic beads, and the functionalized ferromagnetic beads results in formation of complexes, each including one of the functionalized superparamagnetic beads, the analyte, and one of the functionalized ferromagnetic beads, the analyte detectable by co-localization of the functionalized superparamagnetic bead and the functionalized ferromagnetic bead. Contact between a sample not containing the analyte in a sample solution, the functionalized superparamagnetic beads, and the functionalized ferromagnetic beads results in a magnetic interaction energy Uᵢₙₜ between the functionalized superparamagnetic beads and the functionalized ferromagnetic beads, the magnetic interaction energy Uᵢₙₜ being less than or equal to 5k_{B}T, where k_{B} is the Boltzmann constant and T is the temperature of the sample solution.

Example 2 includes the subject matter of Example 1, wherein the diameter d_{A} is in a range of between about 0.1 μm and about 10 μm.

Example 3 includes the subject matter of Example 2, wherein the diameter d_{A} is in a range of between about 0.3 μm and about 3 μm.

Example 4 includes the subject matter of Example 3, wherein the diameter d_{A} is in a range of between about 0.5 μm and about 2 μm.

Example 5 includes the subject matter of Example 4, wherein the diameter d_{A} about 1 μm.

Example 6 includes the subject matter of any of Examples 1-5, wherein the volume magnetic susceptibility X_{A} is in a range of between about 0.01 and about 10.

Example 7 includes the subject matter of Example 6, wherein the volume magnetic susceptibility X_{A} is in a range of between about 0.1 and about 5.

Example 8 includes the subject matter of Example 7, wherein the volume magnetic susceptibility X_{A} is in a range of between about 0.5 and about 3.

Example 9 includes the subject matter of Example 7, wherein the volume magnetic susceptibility X_{A} is about 1.37.

Example 10 includes the subject matter of any of Examples 1-9, wherein the diameter d_{A} is in a range of between about 0.1 μm and about 10 μm.

Example 11 includes the subject matter of Example 10, wherein the diameter d_{B} is in a range of between about 0.3 μm and about 3 μm.

Example 12 includes the subject matter of Example 11, wherein the diameter d_{B} is in a range of between about 0.5 μm and about 2 μm.

Example 13 includes the subject matter of Example 12, wherein the diameter d_{B} about 1.8 μm.

Example 14 includes the subject matter of any of Examples 1-13, wherein the magnetic dipole moment p_{B} is in a range of between 0.02·(d_{B}/[μm])³ mA·μm² and

$${( \frac{20 {π}^{2} ( {k}_{B} T / [ J ] ) {( {d}_{A} / [ μm ] )}^{3}}{( {μ}_{0} / [ \frac{J}{m {A}^{2} μ m} ] ) {X}_{A} Q} )}^{\frac{1}{2}} mA \cdot μ {m}^{2} ,$$

where μ₀ is the vacuum permeability, k_{B} is the Boltzmann constant, T is the temperature of the sample solution, and numerical values of Q(d_{A},d_{B}) are tabulated in Table 1.

Example 15 includes the subject matter of Example 14, wherein the magnetic dipole moment p_{B} is in a range of between 0.2·(d_{B}/[μm])³ mA·μm² and

$${( \frac{20 {π}^{2} ( {k}_{B} T / [ J ] ) {( {d}_{A} / [ μ m ] )}^{3}}{( {μ}_{0} / [ \frac{J}{m {A}^{2} μ m} ] ) {X}_{A} Q} )}^{\frac{1}{2}} mA \cdot μ {m}^{2} .$$

Example 16 includes the subject matter of Example 15, wherein the magnetic dipole moment p_{B} is about 1.0 mA·μm².

Example 17 includes the subject matter of Example 1, wherein the diameter d_{A} is in a range of between about 0.1 μm and about 10 μm, the volume magnetic susceptibility X_{A} is in a range of between about 0.01 and about 10, the diameter d_{B} is in a range of between about 0.1 μm and about 10 μm, and the magnetic dipole moment p_{B} is in a range of between 0.02·(d_{B}/[μm])³ mA·μm² and

$${( \frac{20 {π}^{2} ( {k}_{B} T / [ J ] ) {( {d}_{A} / [ μ m ] )}^{3}}{( {μ}_{0} / [ \frac{J}{m {A}^{2} μ m} ] ) {X}_{A} Q} )}^{\frac{1}{2}} mA \cdot μ {m}^{2} ,$$

where μ₀ is the vacuum permeability, k_{B} is the Boltzmann constant, T is the temperature of the sample solution, and numerical values of Q(d_{A},d_{B}) are tabulated in Table 1.

Example 18 includes the subject matter of any of Examples 1-17, wherein each of the functionalized superparamagnetic beads includes a nonmagnetic core and superparamagnetic material distributed substantially uniformly around the nonmagnetic core.

Example 19 includes the subject matter of any of Examples 1-17, wherein each of the functionalized superparamagnetic beads includes superparamagnetic material distributed substantially uniformly throughout a volume of the functionalized superparamagnetic bead.

Example 20 includes the subject matter of any of Examples 1-19, wherein each of the functionalized ferromagnetic beads includes ferromagnetic material concentrated at a core of the functionalized ferromagnetic bead.

Example 21 includes the subject matter of any of Examples 1-19, wherein each of the functionalized ferromagnetic beads includes ferromagnetic material distributed substantially uniformly throughout a volume of the functionalized ferromagnetic bead.

Example 22 includes the subject matter of any of Examples 1-19, wherein each of the functionalized ferromagnetic beads includes ferromagnetic material distributed over a surface of the functionalized ferromagnetic bead.

Example 23 includes the subject matter of any of Examples 1-22, wherein each of the functionalized superparamagnetic beads or the functionalized ferromagnetic beads further includes a nonmagnetic buffer layer around a surface of the bead.

Example 24 includes the subject matter of any of Examples 1-23, wherein each of the first and the second moiety is a receptor, protein, antibody, cell, virus, or nucleic acid sequence.

Example 25 is a system for detecting a complex including an analyte that includes a bead system including a plurality of functionalized superparamagnetic beads having a diameter d_{A} and a volume magnetic susceptibility X_{A}. The functionalized superparamagnetic beads are functionalized to include a first moiety that associates with an analyte under suitable conditions in a sample solution. The system further includes a plurality of functionalized ferromagnetic beads having a diameter d_{B} and a magnetic dipole moment p_{B}. The functionalized ferromagnetic beads are functionalized to include a second moiety that associates with the analyte under suitable conditions in the sample solution. Contact between a sample containing the analyte in the sample solution, the functionalized superparamagnetic beads, and the functionalized ferromagnetic beads results in formation of complexes, each including one of the functionalized superparamagnetic beads, the analyte, and one of the functionalized ferromagnetic beads, the analyte detectable by co-localization of the functionalized superparamagnetic bead and the functionalized ferromagnetic bead. Contact between a sample not containing the analyte in a sample solution, the functionalized superparamagnetic beads, and the functionalized ferromagnetic beads results in a magnetic interaction energy Uᵢₙₜ between the functionalized superparamagnetic beads and the functionalized ferromagnetic beads, the magnetic interaction energy Uᵢₙₜ being less than or equal to 5k_{B}T, where k_{B} is the Boltzmann constant and T is the temperature of the sample solution. The system further includes a detection apparatus for detecting complexes including the analyte by detecting co-localization of the functionalized superparamagnetic beads and the functionalized ferromagnetic beads.

Example 26 includes the subject matter of Example 25, wherein the detection apparatus includes a substrate, on which the sample can be placed, the substrate including at least one optically detected magnetic resonance (ODMR) center, a light source configured to generate incident light that excites electrons within the at least one ODMR center from a ground state to an excited state, a magnet for applying a bias magnetic field on the complex disposed over the at least one ODMR center, a microwave source configured to generate a microwave field incident on the at least one ODMR center, the microwave source being further configured to generate the microwave field with frequencies that correspond to ground state transitions in the at least one ODMR center, in which the at least one ODMR center produces emitted light when illuminated by the incident light, characteristics of the emitted light being influenced by the microwave field and by the magnetic functionalized beads associated with the analyte in the complex, and an optical photodetector that detects light emitted by the at least one ODMR center.

Example 27 includes the subject matter of Example 26, wherein the at least one ODMR center is a silicon vacancy center in a silicon carbide lattice in the substrate.

Example 28 includes the subject matter of Example 26, wherein the at least one ODMR center is a silicon vacancy center in a diamond lattice in the substrate.

Example 29 includes the subject matter of Example 26, wherein the at least one ODMR center is a nitrogen-vacancy center in a diamond lattice in the substrate.

Example 30 includes the subject matter of any of Examples 26-29, wherein the at least one ODMR center is formed in an upper surface of the substrate.

Example 31 includes the subject matter of any of Examples 26-30, wherein the at least one ODMR center is a plurality of ODMR centers formed in an upper surface of the substrate.

Example 32 includes the subject matter of Example 31, wherein the optical photodetector is an optical imaging system having an imaging sensor that images the emitted light from the plurality of ODMR centers.

Example 33 is a method of detecting a complex including an analyte includes contacting a sample solution containing an analyte with a population of functionalized superparamagnetic beads, which are functionalized to include a first moiety that associates with the analyte under suitable conditions, and contacting the sample solution with a population of functionalized ferromagnetic beads, which are functionalized to include a second moiety that associates with the analyte under suitable conditions, contact of the sample solution with the population of functionalized superparamagnetic beads and the population of functionalized ferromagnetic beads results information of complexes, when the analyte is present in the sample solution, each complex including one of the functionalized superparamagnetic beads, the analyte, and one of the functionalized ferromagnetic beads, or a magnetic interaction energy Uᵢₙₜ between the functionalized superparamagnetic beads and the functionalized ferromagnetic beads in the absence of the analyte in the sample solution, the magnetic interaction energy Uᵢₙₜ being less than or equal to 5k_{B}T, where k_{B} is the Boltzmann constant and T is the temperature of the sample solution, and detecting the complex including the analyte by detecting co-localization of the functionalized superparamagnetic bead and the functionalized ferromagnetic bead.

Example 34 includes the subject matter of Example 33, further including applying a magnetic field gradient to the sample solution after contacting the sample with the population of functionalized superparamagnetic beads.

Example 35 includes the subject matter of Example 34, wherein applying the magnetic field gradient to the sample solution is performed after contacting the sample solution with the population of functionalized ferromagnetic beads.

Example 36 includes the subject matter of Example 33, wherein the population of functionalized superparamagnetic beads and the population of functionalized ferromagnetic beads are added to the sample solution sequentially.

Example 37 includes the subject matter of Example 33, wherein the population of functionalized superparamagnetic beads and the population of functionalized ferromagnetic beads are added to the sample solution simultaneously.

Example 38 includes the subject matter of Example 33, further including applying a magnetic field gradient to the sample solution after contacting the sample solution with the functionalized superparamagnetic and ferromagnetic beads.

Example 39 includes the subject matter of any of Examples 34-38, further including varying the magnetic field gradient applied to the sample solution.

Example 40 includes the subject matter of any of Examples 33-39, further including concentrating the sample solution after contacting the sample solution with the population of functionalized ferromagnetic beads.

Example 41 includes the subject matter of any of Examples 33-40, further including agglomerating a plurality of functionalized superparamagnetic and ferromagnetic beads, after contacting the sample solution with the population of functionalized ferromagnetic beads, before detecting the complex.

Example 42 includes the subject matter of any of Examples 33-41, further including disposing the sample solution potentially including the complex over a substrate that includes at least one optically detected magnetic resonance (ODMR) center formed in the substrate; exciting electrons within the at least one ODMR center from a ground state to an excited state with incident light; applying a bias magnetic field on the complex; and generating a microwave field incident on the at least one ODMR center, the microwave field including frequencies that correspond to ground state transitions in the at least one ODMR center, wherein detecting the complex including the analyte further includes analyzing light emitted by the at least one ODMR center, characteristics of the emitted light being influenced by the microwave field and by the functionalized superparamagnetic and ferromagnetic beads associated with the analyte in the complex.

Example 43 includes the subject matter of Example 42, wherein the at least one ODMR center is a nitrogen-vacancy center in a diamond lattice.

Example 44 includes the subject matter of any of Examples 42-43, wherein the at least one ODMR center is formed in an upper surface of the substrate.

Example 45 includes the subject matter of any of Examples 42-44, wherein the at least one ODMR center is a plurality of ODMR centers formed in the upper surface of the substrate.

Example 46 includes the subject matter of any of Examples 42-45, wherein analyzing light emitted by the at least one ODMR center includes imaging the emitted light.

Example 47 includes the subject matter of any of Examples 42-46, further including dehydrating the sample solution after disposing the sample solution over the substrate.

### EQUIVALENTS

Having thus described several illustrative embodiments, it is to be appreciated that various alterations, modifications, and improvements will readily occur to those skilled in the art. Such alterations, modifications, and improvements are intended to form a part of this disclosure and are intended to be within the spirit and scope of this disclosure. While some examples presented herein involve specific combinations of functions or structural elements, it should be understood that those functions and elements may be combined in other ways according to the present disclosure to accomplish the same or different objectives. In particular, acts, elements, and features discussed in connection with one embodiment are not intended to be excluded from similar or other roles in other embodiments. Additionally, elements and components described herein may be further divided into additional components or joined together to form fewer components for performing the same functions.

The foregoing description of example embodiments has been presented for the purposes of illustration and description. It is not intended to be exhaustive or to limit the present disclosure to the precise forms disclosed. Many modifications and variations are possible in light of this disclosure. It is intended that the scope of the present disclosure be limited not by this detailed description, but rather by the claims appended hereto. Future filed applications claiming priority to this application may claim the disclosed subject matter in a different manner and may generally include any set of one or more limitations as variously disclosed or otherwise demonstrated herein.

What we claim is:

1. A bead system for bead-based analyte detection, the bead system comprising:
(a) a plurality of functionalized superparamagnetic beads having a diameter d_{A} and a volume magnetic susceptibility X_{A}, the plurality of functionalized superparamagnetic beads functionalized to include a first moiety that associates with an analyte under suitable conditions in a sample solution; and
(b) a plurality of functionalized ferromagnetic beads having a diameter d_{B} and a magnetic dipole moment p_{B}, the plurality of functionalized ferromagnetic beads functionalized to include a second moiety that associates with the analyte under suitable conditions in the sample solution,
wherein contact between a sample containing the analyte in the sample solution, the functionalized superparamagnetic beads, and the functionalized ferromagnetic beads results in formation of complexes, each including one of the functionalized superparamagnetic beads, the analyte, and one of the functionalized ferromagnetic beads, the analyte detectable by co-localization of the functionalized superparamagnetic bead and the functionalized ferromagnetic bead,
wherein contact between a sample not containing the analyte in a sample solution, the functionalized superparamagnetic beads, and the functionalized ferromagnetic beads results in a magnetic interaction energy Uᵢₙₜ between the functionalized superparamagnetic beads and the functionalized ferromagnetic beads, the magnetic interaction energy Uᵢₙₜ being less than or equal to 5k_{B}T, where k_{B} is the Boltzmann constant and T is the temperature of the sample solution.

2. The bead system of claim 1, wherein the diameter d_{A} is in a range of between about 0.1 μm and about 10 μm.

3. The bead system of claim 2, wherein the diameter d_{A} is in a range of between about 0.3 μm and about 3 μm.

4. The bead system of claim 3, wherein the diameter d_{A} is in a range of between about 0.5 μm and about 2 μm.

5. The bead system of claim 4, wherein the diameter d_{A} is about 1 μm.

6. The bead system of claim 1, wherein the volume magnetic susceptibility X_{A} is in a range of between about 0.01 and about 10.

7. The bead system of claim 6, wherein the volume magnetic susceptibility X_{A} is in a range of between about 0.1 and about 5.

8. The bead system of claim 7, wherein the volume magnetic susceptibility X_{A} is in a range of between about 0.5 and about 3.

9. The bead system of claim 8, wherein the volume magnetic susceptibility X_{A} is about 1.37.

10. The bead system of claim 1, wherein the diameter d_{B} is in a range of between about 0.1 μm and about 10 μm.

11. The bead system of claim 10, wherein the diameter d_{B} is in a range of between about 0.3 μm and about 3 μm.

12. The bead system of claim 11, wherein the diameter d_{B} is in a range of between about 0.5 μm and about 2 μm.

13. The bead system of claim 12, wherein the diameter d_{B} is about 1.8 μm.

14. The bead system of claim 1, wherein the magnetic dipole moment p_{B} is in a range of between 0.02·(d_{B}/[μm])³ mA·μm² and
$${( \frac{20 {π}^{2} ( {k}_{B} T / [ J ] ) {( {d}_{A} / [ μ m ] )}^{3}}{( {μ}_{0} / [ \frac{J}{m {A}^{2} μ m} ] ) {X}_{A} Q} )}^{\frac{1}{2}} mA \cdot μ {m}^{2} ,$$
where μ₀ is the vacuum permeability, k_{B} is the Boltzmann constant, T is the temperature of the sample solution, and numerical values of Q(d_{A},d_{B}) are tabulated in Table 1.

15. The bead system of claim 14, wherein the magnetic dipole moment p_{B} is in a range of between 0.2·(d_{B}/[μm])³ mA·μm² and
$${( \frac{20 {π}^{2} ( {k}_{B} T / [ J ] ) {( {d}_{A} / [ μ m ] )}^{3}}{( {μ}_{0} / [ \frac{J}{m {A}^{2} μ m} ] ) {X}_{A} Q} )}^{\frac{1}{2}} mA \cdot μ {m}^{2} .$$

16. The bead system of claim 15, wherein the magnetic dipole moment p_{B} is about 1.0 mA·μm².

17. The bead system of claim 1, wherein the diameter d_{A} is in a range of between about 0.1 μm and about 10 μm, the volume magnetic susceptibility X_{A} is in a range of between about 0.01 and about 10, the diameter d_{B} is in a range of between about 0.1 μm and about 10 μm, and the magnetic dipole moment p_{B} is in a range of between 0.02·(d_{B}/[μm])³ mA·μm² and
$${( \frac{20 {π}^{2} ( {k}_{B} T / [ J ] ) {( {d}_{A} / [ μ m ] )}^{3}}{( {μ}_{0} / [ \frac{J}{m {A}^{2} μ m} ] ) {X}_{A} Q} )}^{\frac{1}{2}} mA \cdot μ {m}^{2} ,$$
where μ₀ is the vacuum permeability, k_{B} is the Boltzmann constant, T is the temperature of the sample solution, and numerical values of Q(d_{A},d_{B}) are tabulated in Table 1.

18. The bead system of claim 1, wherein each of the functionalized superparamagnetic beads includes a nonmagnetic core and superparamagnetic material distributed substantially uniformly around the nonmagnetic core.

19. The bead system of claim 1, wherein each of the functionalized superparamagnetic beads includes superparamagnetic material distributed substantially uniformly throughout a volume of the functionalized superparamagnetic bead.

20. The bead system of claim 1, wherein each of the functionalized ferromagnetic beads includes ferromagnetic material concentrated at a core of the functionalized ferromagnetic bead.

21. The bead system of claim 1, wherein each of the functionalized ferromagnetic beads includes ferromagnetic material distributed substantially uniformly throughout a volume of the functionalized ferromagnetic bead.

22. The bead system of claim 1, wherein each of the functionalized ferromagnetic beads includes ferromagnetic material distributed over a surface of the functionalized ferromagnetic bead.

23. The bead system of claim 1, wherein each of the functionalized superparamagnetic beads or the functionalized ferromagnetic beads further includes a nonmagnetic buffer layer around a surface of the bead.

24. The bead system of claim 1, wherein each of the first and the second moiety is a receptor, protein, antibody, cell, virus, or nucleic acid sequence.

25. A system for detecting a complex including an analyte, the system comprising:
(a) a bead system comprising:
(i) a plurality of functionalized superparamagnetic beads having a diameter d_{A} and a volume magnetic susceptibility X_{A}, the plurality of functionalized superparamagnetic beads functionalized to include a first moiety that associates with an analyte under suitable conditions in a sample solution; and
(ii) a plurality of functionalized ferromagnetic beads having a diameter d_{B} and a magnetic dipole moment p_{B}, the plurality of functionalized ferromagnetic beads functionalized to include a second moiety that associates with the analyte under suitable conditions in the sample solution,
wherein contact between a sample containing the analyte in the sample solution, the functionalized superparamagnetic beads, and the functionalized ferromagnetic beads results in formation of complexes, each including one of the functionalized superparamagnetic beads, the analyte, and one of the functionalized ferromagnetic beads, the analyte detectable by co-localization of the functionalized super-paramagnetic bead and the functionalized ferromagnetic bead,
wherein contact between a sample not containing the analyte in a sample solution, the functionalized superparamagnetic beads, and the functionalized ferromagnetic beads results in a magnetic interaction energy Uᵢₙₜ between the functionalized superparamagnetic beads and the functionalized ferromagnetic beads, the magnetic interaction energy Uᵢₙₜ being less than or equal to 5k_{B}T, where k_{B} is the Boltzmann constant and T is the temperature of the sample solution; and
(b) a detection apparatus for detecting complexes including the analyte by detecting co-localization of the functionalized superparamagnetic beads and the functionalized ferromagnetic beads.

26. The system of claim 25, wherein the detection apparatus comprises:
(i) a substrate, on which the sample can be placed, the substrate including at least one optically detected magnetic resonance (ODMR) center;
(ii) a light source configured to generate incident light that excites electrons within the at least one ODMR center from a ground state to an excited state;
(iii) a magnet for applying a bias magnetic field on the complex disposed over the at least one ODMR center;
(iv) a microwave source configured to generate a microwave field incident on the at least one ODMR center, the microwave source being further configured to generate the microwave field with frequencies which correspond to ground state transitions in the at least one ODMR center, in which the at least one ODMR center produces emitted light when illuminated by the incident light, characteristics of the emitted light being influenced by the microwave field and by the magnetic functionalized beads associated with the analyte in the complex; and
(v) an optical photodetector that detects light emitted by the at least one ODMR center.

27. The system of claim 26, wherein the at least one ODMR center is a silicon vacancy center in a silicon carbide lattice in the substrate.

28. The system of claim 26, wherein the at least one ODMR center is a silicon vacancy center in a diamond lattice in the substrate.

29. The system of claim 26, wherein the at least one ODMR center is a nitrogen-vacancy center in a diamond lattice in the substrate.

30. The system of claim 29, wherein the at least one ODMR center is formed in an upper surface of the substrate.

31. The system of claim 30, wherein the at least one ODMR center is a plurality of ODMR centers formed in the upper surface of the substrate.

32. The system of claim 31, wherein the optical photodetector is an optical imaging system having an imaging sensor that images the emitted light from the plurality of ODMR centers.

33. A method of detecting a complex including an analyte, the method comprising:
(a) contacting a sample solution potentially containing an analyte with a population of functionalized superparamagnetic beads, which are functionalized to include a first moiety that associates with the analyte under suitable conditions;
(b) contacting the sample solution with a population of functionalized ferromagnetic beads, which are functionalized to include a second moiety that associates with the analyte under suitable conditions; and
wherein contact of the sample solution with the population of functionalized superparamagnetic beads and the population of functionalized ferromagnetic beads results in:
i) formation of complexes, when the analyte is present in the sample solution, each complex including one of the functionalized superparamagnetic beads, the analyte, and one of the functionalized ferromagnetic beads; or
ii) a magnetic interaction energy Uᵢₙₜ between the functionalized superparamagnetic beads and the functionalized ferromagnetic beads in the absence of the analyte in the sample solution, the magnetic interaction energy Uᵢₙₜ being less than or equal to 5k_{B}T, where k_{B} is the Boltzmann constant and T is the temperature of the sample solution;
(c) detecting the complex including the analyte by detecting co-localization of the functionalized superparamagnetic bead and the functionalized ferromagnetic bead.

34. The method of claim 33, further including applying a magnetic field gradient to the sample solution after contacting the sample with the population of functionalized superparamagnetic beads.

35. The method of claim 34, wherein applying the magnetic field gradient to the sample solution is performed after contacting the sample solution with the population of functionalized ferromagnetic beads.

36. The method of claim 33, wherein the population of functionalized superparamagnetic beads and the population of functionalized ferromagnetic beads are added to the sample solution sequentially.

37. The method of claim 33, wherein the population of functionalized superparamagnetic beads and the population of functionalized ferromagnetic beads are added to the sample solution simultaneously.

38. The method of claim 33, further including applying a magnetic field gradient to the sample solution after contacting the sample solution with the functionalized superparamagnetic and ferromagnetic beads.

39. The method of claim 38, further including varying the magnetic field gradient applied to the sample solution.

40. The method of claim 33, further including concentrating the sample solution after contacting the sample solution with the population of functionalized ferromagnetic beads.

41. The method of claim 33, further including agglomerating a plurality of functionalized superparamagnetic and ferromagnetic beads, after contacting the sample solution with the population of functionalized ferromagnetic beads, before detecting the complex.

42. The method of claim 33, wherein detecting the complex includes disposing the sample solution potentially including the complex over a substrate that includes at least one optically detected magnetic resonance (ODMR) center formed in the substrate; exciting electrons within the at least one ODMR center from a ground state to an excited state with incident light; applying a bias magnetic field on the complex; generating a microwave field incident on the at least one ODMR center, the microwave field including frequencies that correspond to ground state transitions in the at least one ODMR center, and analyzing light emitted by the at least one ODMR center, characteristics of the emitted light being influenced by the microwave field and by the functionalized superparamagnetic and ferromagnetic beads associated with the analyte in the complex.

43. The method of claim 42, wherein the at least one ODMR center is a nitrogen-vacancy center in a diamond lattice in the substrate.

44. The method of claim 43, wherein the at least one ODMR center is formed in an upper surface of the substrate.

45. The method of claim 44, wherein the at least one ODMR center is a plurality of ODMR centers formed in the upper surface of the substrate.

46. The method of claim 45, wherein analyzing light emitted by the plurality of ODMR centers includes imaging the emitted light.

47. The method of claim 42, further including dehydrating the sample solution after disposing the sample solution over the substrate.
